(19) United States
(12) Patent Application Publication
(10) Pub. No.: US 2024/0269234 A1
(43) Pub. Date: Aug. 15, 2024

(54) METHOD OF TREATING CEREBRAL AUTOSOMAL DOMINANT ARTERIOPATHY WITH SUBCORTICAL INFARCT AND LEUKOENCEPHALOPATHY (CADASIL)

(71) Applicant: The Research Foundation for the State University of New York, Albany, NY (US)

(72) Inventors: Li-Ru Zhao, Fayetteville, NY (US); Suning Ping, Syracuse, NY (US)

(21) Appl. No.: 18/646,262

(22) Filed: Apr. 25, 2024

Related U.S. Application Data
(63) Continuation of application No. 17/390,453, filed on Jul. 30, 2021, now abandoned.
(60) Provisional application No. 63/059,159, filed on Jul. 30, 2020.

(51) Int. Cl. A61K 38/19; A61K 38/18; A61P 25/28

(52) U.S. Cl. CPC A61K 38/193; A61K 38/1866; A61K 38/19; A61P 25/28

(57) ABSTRACT

The present disclosure relates to a method of improving neurological function and enhancing cerebrovascular maintenance and regeneration in a mammal suffering from cerebral autosomal dominant arteriopathy with subcortical infarcts and leukoencephalopathy (CADASIL), including administering an effective amount of a composition including a granulocyte colony-stimulating factor (G-CSF) polypeptide in combination with a stem cell factor (SCF) polypeptide.

### CROSS-REFERENCE TO RELATED APPLICATIONS

The present disclosure claims priority or the benefit under 35 U.S.C. § 119 of U.S. provisional application No. 63/059,159 filed Jul. 30, 2020, herein entirely incorporated by reference.

### REFERENCE TO A SEQUENCE LISTING

This application contains a Sequence Listing in computer readable form, which is incorporated herein by reference.

### FIELD OF THE INVENTION

The present disclosure relates generally to the field of neurology and neurobiology. More specifically, the present disclosure relates to the use of vascular endothelial growth factor (VEGF) to treat, ameliorate, or prevent cerebral autosomal dominant arteriopathy with subcortical infarcts and leukoencephalopathy (CADASIL). In embodiments, stem cell factor (SCF) polypeptide alone, or in combination with granulocyte colony-stimulating factor (G-CSF) polypeptide is administered to target or increase vascular endothelial growth factor (VEGF, such as VEGF-A) to treat, ameliorate, or prevent CADASIL.

### BACKGROUND

Cerebral autosomal dominant arteriopathy with subcortical infarcts and leukoencephalopathy (CADASIL) is a common monogenic cause of cerebral small vessel disease and represents a frequent form of hereditary ischemic stroke and vascular dementia in adults. (See e.g., Chabriat H, et al., (2009) Cadasil, Lancet Neurol 8 (7):643-653). CADASIL mainly affects young and middle-aged adults and causes severe disability and early death. (See e.g., Chabriat H, et al., (1995) Cerebral autosomal dominant arteriopathy with subcortical infarcts and leukoencephalopathy: a positron emission tomography study in two affected family members. Stroke 26 (9): 1729-1730; Peters N, et al., (2004) A two-year clinical follow-up study in 80 CADASIL subjects: progression patterns and implications for clinical trials. Stroke 35 (7):1603-1608; Peters N, et al., (2004) CADASIL associated Notch3 mutations have differential effects both on ligand binding and ligand-induced Notch3 receptor signaling through RBP-Jk. Exp Cell Res 299 (2):454-464; Di Donato I, et al., (2017) Cerebral Autosomal Dominant Arteriopathy with Subcortical Infarcts and Leukoencephalopathy (CADASIL) as a model of small vessel disease: update on clinical, diagnostic, and management aspects. BMC Med 15 (1):41; and Joutel A (2011) Pathogenesis of CADASIL: transgenic and knock-out mice to probe function and dysfunction of the mutated gene, Notch3, in the cerebrovasculature Bioessays 33 (1): 73-80). It has long been thought that CADASIL is a rare genetic disease. However, a recent genetic study challenged this notion by revealing the prevalence of CADASIL mutations is 3.4/1000 in the world, which is 100 times higher than previously thought. (See e.g., Rutten et al., Archetypal NOTCH3 mutations frequent in public exome: implications for CADASIL. Ann Clin Transl Neurol 3 (11), 844-853 (2016)).

NOTCH3 gene mutation in vascular smooth muscle cells (VSMCs) of small arteries and pericytes of capillaries is the cause of this genetic disease. The causative role of NOTCH3 gene mutation in CADASIL was previously discovered. (See e.g., Joutel A, et al., (1996) Notch3 mutations in CADASIL, a hereditary adult-onset condition causing stroke and dementia. Nature 383 (6602):707-710). Since then, tremendous effort has been made to understand the pathology of CADASIL. However, it remains largely unknown how NOTCH3 gene mutation drives CADASIL progression. No treatment is currently available for CADASIL. (See e.g., Di Donato I, et al., (2017) Cerebral Autosomal Dominant Arteriopathy with Subcortical Infarcts and Leukoencephalopathy (CADASIL) as a model of small vessel disease: update on clinical, diagnostic, and management aspects. BMC Med 15 (1): 41). The lack of knowledge of the molecular mechanisms underlying the pathogenesis of CADASIL builds a barrier that makes it difficult to find a therapeutic target to stop or delay the disease progression.

VSMCs in small arteries and pericytes in capillaries belong to mural cells embedded within the basal lamina of blood vessels. CADASIL-related NOTCH3 gene missense mutations affect the epidermal growth factor-like repeats (EGFr) in the extracellular domain of Notch3 receptor (Notch3ECD). (See e.g., Joutel A (2011) Pathogenesis of CADASIL: transgenic and knock-out mice to probe function and dysfunction of the mutated gene, Notch3, in the cerebrovasculature. Bioessays 33 (1):73-80; and Joutel A, et al., (1997) Strong clustering and stereotyped nature of Notch3 mutations in CADASIL patients. Lancet 350 (9090): 1511-1515). The accumulation of Notch3ECD and the deposits of granular osmiophilic material (GOM) close to the cell surface of mural cells are the two hallmarks of CADASIL pathology. (See e.g., Joutel A (2011) Pathogenesis of CADASIL: transgenic and knock-out mice to probe function and dysfunction of the mutated gene, Notch3, in the cerebrovasculature. Bioessays 33 (1): 73-80; and Joutel A (2015) The NOTCH3ECD cascade hypothesis of cerebral autosomal-dominant arteriopathy with subcortical infarcts and leukoencephalopathy disease. Neurology and Clinical Neuroscience 3 (1): 1-6). The vast majority of CADASIL patients have Notch3ECD mutations in the EGFr 2-5. (See e.g., Baudrimont M, et al., (1993) Autosomal dominant leukoencephalopathy and subcortical ischemic stroke. A clinicopathological study. Stroke 24 (1): 122-125; and Masek J, Andersson E R (2017) The developmental biology of genetic Notch disorders. Development 144 (10): 1743-1763).

The R90C mutation is located in the EGFr 2 (See e.g., Monet M, et al., (2007) The archetypal R90C CADASIL-NOTCH3 mutation retains NOTCH3 function in vivo. Hum Mol Genet16 (8): 982-992), indicating that R90C mutation is one of the common forms of CADASIL. In the transgenic mouse model of Notch3ECD-R90C mutation (TgNotch3R90C mice), CADASIL-related pathology has been observed, including age-dependent CADASIL-associated vascular pathology such as VSMC/pericyte degeneration (See e.g., Ruchoux M M, et al., (2003) Transgenic mice expressing mutant Notch3 develop vascular alterations characteristic of cerebral autosomal dominant arteriopathy with subcortical infarcts and leukoencephalopathy. The American journal of pathology 162 (1):329-342; and Gu X, et al., (2012) Ultrastructural changes in cerebral capillary pericytes in aged Notch3 mutant transgenic mice. Ultrastruct Pathol 36 (1):48-55), cerebrovascular dysfunction (See e.g., Lacombe P, et al., (2005) Impaired cerebral vasoreactivity in a transgenic mouse model of cerebral autosomal dominant arteriopathy with subcortical infarcts and leukoencephalopathy arteriopathy. Stroke 36 (5):1053-1058), Notch3ECD/GOM aggregation (See e.g., Ruchoux M M, et al., (2003) Transgenic mice expressing mutant Notch3 develop vascular alterations characteristic of cerebral autosomal dominant arteriopathy with subcortical infarcts and leukoencephalopathy. The American journal of pathology 162 (1):329-342), cognitive decline (See e.g., Liu X Y, et al., (2015) Stem cell factor and granulocyte colony-stimulating factor exhibit therapeutic effects in a mouse model of CADASIL. Neurobiol Dis 73:189-203), cerebral capillary thrombosis (See Ping S, et al., (2018) Stem Cell Factor in Combination with Granulocyte Colony-Stimulating Factor reduces Cerebral Capillary Thrombosis in a Mouse Model of CADASIL. Cell Transplant 27 (4):637 647), and cerebral small infarcts (See e.g., Ruchoux M M, et al., (2003) Transgenic mice expressing mutant Notch3 develop vascular alterations characteristic of cerebral autosomal dominant arteriopathy with subcortical infarcts and leukoencephalopathy. The American journal of pathology 162 (1):329-342). In TgNotch3R90C mice, degeneration of VSMCs and cerebrovascular dysfunction with reduced cerebral blood flow occur at the age of 10 months. (See e.g., Ruchoux M M, et al., (2003) Transgenic mice expressing mutant Notch3 develop vascular alterations characteristic of cerebral autosomal dominant arteriopathy with subcortical infarcts and leukoencephalopathy. The American journal of pathology 162 (1):329-342), and Lacombe P, et al., (2005) Impaired cerebral vasoreactivity in a transgenic mouse model of cerebral autosomal dominant arteriopathy with subcortical infarcts and leukoencephalopathy arteriopathy. Stroke 36 (5): 1053-1058).

Prior art of interest includes U.S. Pat. No. 20060153799, entitled Use of SCF and G-CSF in the treatment of cerebral ischemia and neurological disorders. However, the prior art fails to target VEGF in accordance with the methods of the present disclosure.

The inventors have observed that deficient vascular endothelial growth factors (VEGF), such as in mural cells, plays an essential role in the development of CADASIL.

Accordingly, there is a continuing need for methods for bolstering VEGF function quantity, or performance to treat or alleviate pathology and symptoms relating to CADASIL.

### SUMMARY

In some embodiments, the present disclosure relates to a method of improving neurological function, cerebral blood flow, and/or enhancing cerebrovascular maintenance and regeneration in a mammal suffering from cerebral autosomal dominant arteriopathy with subcortical infarcts and leukoencephalopathy (CADASIL).

In some embodiments, methods and compositions for treating or ameliorating cerebral autosomal dominant arteriopathy with subcortical infarcts and leukoencephalopathy (CADASIL), include administering vascular endothelial growth factor (VEGF), or functional isoforms thereof, to a brain of a subject in need thereof. Non-limiting examples of suitable VEGF, or functional isoforms thereof, suitable for use herein include one or more of VEGFa, VEGFb, VEDFc, or combinations thereof. In embodiments, pharmaceutically acceptable forms and pharmaceutically acceptable salt forms of VEGF are suitable for use herein.

In some embodiments, the present disclosure relates to a method of improving neurological function in a mammal suffering from cerebral autosomal dominant arteriopathy with subcortical infarcts and leukoencephalopathy (CADASIL), including administering an effective amount of a composition including VEGF or isoforms thereof. In embodiments, pharmaceutically acceptable compositions are suitable for use herein. In embodiments, an effective amount is a therapeutically acceptable amount.

In some embodiments, the present disclosure relates to a method of improving neurological function in a mammal suffering from cerebral autosomal dominant arteriopathy with subcortical infarcts and leukoencephalopathy (CADASIL), including administering an effective amount of a composition including one or more of VEGF, a granulocyte colony-stimulating factor (G-CSF) polypeptide, a stem cell factor (SCF) polypeptide, or combinations thereof. In embodiments, an effective amount is a therapeutically acceptable amount.

In some embodiments, the present disclosure relates to a method of improving neurological function and cerebral blood flow and enhancing cerebrovascular maintenance and regeneration in a mammal suffering from cerebral autosomal dominant arteriopathy with subcortical infarcts and leukoencephalopathy (CADASIL), including methods and compositions for treating or ameliorating cerebral autosomal dominant arteriopathy with subcortical infarcts and leukoencephalopathy (CADASIL), including administering an effective amount of a granulocyte colony-stimulating factor (G-CSF) polypeptide and a stem cell factor (SCF) polypeptide to a subject in need thereof, or, in some embodiments administering a composition including a granulocyte colony-stimulating factor (G-CSF) polypeptide and a stem cell factor (SCF) polypeptide.

In embodiments, the present disclosure relates to a method of improving neurological function and cerebral blood flow and enhancing cerebrovascular maintenance and regeneration in a mammal suffering from CADASIL, including administering an effective amount of a composition including a stem cell factor (SCF) polypeptide alone, or in combination with granulocyte colony-stimulating factor (G-CSF) polypeptide.

In some embodiments, the present disclosure relates to a method of improving neurological function and cerebral blood flow and enhancing cerebrovascular maintenance and regeneration in a mammal suffering from CADASIL, including administering an effective amount of a composition including a granulocyte colony-stimulating factor (G-CSF) polypeptide alone or in combination with a stem cell factor (SCF) polypeptide.

In some embodiments, the present disclosure relates to a method of improving neurological function in a mammal suffering from cerebral autosomal dominant arteriopathy with subcortical infarcts and leukoencephalopathy (CADASIL), including administering an effective amount of a composition including a granulocyte colony-stimulating factor (G-CSF) polypeptide in combination with a stem cell factor (SCF) polypeptide.

In embodiments, the present disclosure relates to a method of improving neurological function in a mammal suffering from cerebral autosomal dominant arteriopathy with subcortical infarcts and leukoencephalopathy (CADASIL), including administering an effective amount of a composition including a stem cell factor (SCF) polypeptide, alone or in combination with granulocyte colony-stimulating factor (G-CSF) polypeptide.

In some embodiments, the present disclosure relates to a method of improving neurological function in a mammal suffering from cerebral autosomal dominant arteriopathy with subcortical infarcts and leukoencephalopathy (CADASIL), including administering an effective amount of a composition including a granulocyte colony-stimulating factor (G-CSF) polypeptide alone or in combination with a stem cell factor (SCF) polypeptide.

The illustrative aspects of the present disclosure are designed to solve the problems herein described and/or other problems not discussed.

### BRIEF DESCRIPTION OF THE DRAWINGS, AND SEQUENCE LISTINGS

The patent or application file contains at least one drawing executed in color. Copies of this patent or patent application publication with color drawing(s) will be provided by the Office upon request and payment of the necessary fee.

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1 depicts a flow diagram of a method for improving neurological function and cerebral blood flow and enhancing cerebrovascular maintenance and regeneration in a mammal suffering from CADASIL in accordance with the present disclosure.

FIG. 2 depicts a flow diagram of a method for improving neurological function and cerebral blood flow and enhancing cerebrovascular maintenance and regeneration in a mammal suffering from CADASIL, in accordance with the present disclosure.

FIG. 3 depicts a flow diagram of a method for improving neurological function and cerebral blood flow and enhancing cerebrovascular maintenance and regeneration in a mammal suffering from CADASIL, in accordance with the present disclosure.

FIGS. 4A and 4B) are histograms showing secreted VEGF-A in vascular smooth muscle cells (VSMCs) and pericytes by enzyme-linked immunosorbent assay (ELISA). Note that the levels of VEGF-A secreted from VSMCs and pericytes are significantly decreased in these mural cells isolated from 3-month-old TgNotch3R90C mice (Tg) as compared to age-matched wild type (WT) mice. **p<0.01. Independently repeated for 3 times.

FIGS. 5A-5F depict immunohistochemistry data revealing the decreased VEGF expression (green) in both vascular smooth muscle cells (red, alpha.SMA positive cells) and pericytes (red, CD13 positive cells) in the brains of 3-month-old TgNotch3R90C mice. FIGS. 5A-D depict representative confocal images. Blue: DAPI nuclear counterstain. FIGS. 5E-5F depict quantification data. ***p<0.001. WT: age matched wild type mice. TgNothch3: TgNotch3R90C mice.

FIGS. 6A-6C depict changes of VEGF-A and its receptor, VEGFR2, in vascular smooth muscle cells (VSMCs). VSMCs were isolated from the brains of 3-month-old wild type (WT) mice or TgNotch3R90C (TgNotch3) mice. FIG. 6A depicts Western blot images. FIGS. 6B and 6C depict two histograms. Data show that the protein levels of VEGF-A and VEGFR2 are significantly decreased in VSMCs of TgNotch3R90C mice. The decreased VEGFR2 activity and VEGF-A in VSMCs of TgNotch3R90C mice are restored by SCF+G-CSF treatment. *p<0.05. Independently repeated for 3 times.

FIGS. 7A-7C show that VEGF-A is required for survival of VSMCs with Notch3R90C mutation (i.e. CADASIL-associated mutation). FIG. 7A depicts Western blot analysis. Reduced VEGF-A is seen in VSMCs of TgNotch3R90C mice (i.e. Tg-VSMCs). siRNA against VEGF-A (siR-VEGF) knocks down VEGF-A in Tg-VSMCs. FIG. 7B depicts representative images showing live VSMCs (green) and dead VSMCs (red). FIG. 7C depicts a histogram. Note that knocking down VEGF-A in Tg-VSMCs leads to increases of Tg-VSMC death. SCF+G-CSF treatment significantly reduces Tg-VSMC death and increases Tg-VSMC survival. Knocking down VEGF-A in Tg-VSMCs completely blocks the SCF+G-CSF-enhanced Tg-VSMC survival, indicating that VEGF-A is required to enhance Tg-VSMC survival by SCF+G-CSF treatment. VSMCs were isolated from the brains of 3-month-old wild type (WT) mice or TgNotch3R90C (Tg) mice. siR-Con: siRNA control. S+G: SCF+G-CSF treatment. *p<0.05, **p<0.01, ***p<0.001. Independently repeated for 3 times.

FIGS. 8A-8D depict the efficacy of VEGF treatment in preventing Tg-VSMC death. VSMCs were isolated from the brains of 3-month-old wild type (WT) mice or TgNotch3R90C (TgNotch3) mice. FIGS. 8A-8C depict representative immunocytochemistry images showing live VSMCs (green) and dead VSMCs (red) in WT-VSMCs (FIG. A) and the Tg-VSMCs treated with or without VEGF treatment (FIGS. 8B and 8C). FIG. 8D depicts a histogram. Note that Notch3R90C mutation-increased VSMC death is prevented by VEGF treatment. ***p<0.001. Independently repeated for 3 times.

FIG. 9 depicts upstream regulators of affected genes in brain-isolated Tg-VSMCs and regulatory networks of affected genes in brain-isolated Tg-VSMCs. VSMCs were isolated from the brains of 3-month-old wild type (WT) mice or TgNotch3R90C mice. After running RNA sequencing, the upstream regulators and regulatory networks of affected genes in brain-isolated Tg-VSMCs were analyzed using Ingenuity Pathway Analysis software. Note that the vegf gene is identified as one of the most important upstream regulators for Tg-VSMCs. Other important upstream regulators that show tight connections with vegf are p38 MAPK and NF-kB which are also affected in Tg-VSMCs as compared to WT-VSMCs.

FIGS. 10A-10C depict restored ERK and NF-kB signaling by SCF+G-CSF treatment. ERK is the best representative molecule of p38 MAPK. Note that both ERK and NF-kB signaling are decreased in Tg-VSMCs. SCF+G-CSF treatment restores the ERK and NF-kB signaling activation in Tg-VSMCs. VSMCs were isolated from the brains of 3-month-old wild type (WT) mice or TgNotch3R90C (TgNotch3) mice. S+G: SCF+G-CSF treatment. *p<0.05. Independently repeated for 4 times.

FIGS. 11A-11F depict Western blot data showing the efficacy of VEGF in restoring PI3K/AKT, ERK, NF-kB and P38 cell signaling activation in brain-isolated Tg-VSMCs. FIG. 11A depicts Western blot images. FIGS. 11B-11F depict five histograms. Note that PI3K/AKT, ERK, NF-kB and P38 cell signaling activation are decreased in Tg-VSMCs as compared to WT-VSMCs. VEGF treatment restores the PI3K/AKT, ERK, NF-kB and P38 cell signaling activation in Tg-VSMCs. VSMCs were isolated from the brains of 3-5-month-old wild type (WT) mice or TgNotch3R90C (TgNotch3) mice. *p<0.05. Independently repeated for 3 times.

SEQ ID NO: 1 depicts human VEGFa.

SEQ ID NO: 2 depicts a human VEGFa isoform.

SEQ ID NO: 3 depicts a human VEGFa isoform.

SEQ ID NO: 4 depicts mouse VEGFa.

SEQ ID NO: 5 depicts human VEGFb.

SEQ ID NO: 6 depicts human VEGFc.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### DETAILED DESCRIPTION

Embodiments of the present disclosure treat, ameliorate, or eliminate cerebral autosomal dominant arteriopathy with subcortical infarcts and leukoencephalopathy (CADASIL) in subjects in need thereof. For examples, embodiments, of the present disclosure include compositions and methods for preventing, treating, or ameliorating cerebral autosomal dominant arteriopathy with subcortical infarcts and leukoencephalopathy (CADASIL), including administering vascular endothelial growth factor (VEGF), or functional isoforms thereof, to a brain of a subject in need thereof. In embodiments, administering includes any suitable method of increasing the amount of VEGF in a subject in need thereof. In embodiments, VEGF, or functional isoforms thereof, include one or more of VEGFa, VEGFb, VEDFc, or combinations thereof. In embodiments, the VEGF, or functional isoforms thereof, include or consist of an amino acid sequence having at least 90%, 95%, 97%, 99% sequence identity to SEQ ID NO: 1, SEQ ID NO: 2, SEQ ID NO:3, SEQ ID NO:4; SEQ ID NO:5, or SEQ ID NO:6 or a pharmaceutically acceptable salt form thereof. In embodiments, the VEGF, or functional isoforms thereof may have several (e.g., 1-5) conservative substitutions, or alterations that do not alter the function of the peptide.

In embodiments, administering VEGF, or functional isoforms thereof, includes administering one or more pharmaceutically acceptable agents or compositions that increase VEGF in a subject. A non-limiting example of a way to increase VEGF in a subject includes administering a granulocyte colony-stimulating factor (G-CSF) polypeptide, alone, or in combination with a stem cell factor (SCF) polypeptide, in an amount sufficient, such as a therapeutically acceptable amount, to increase VEGF in a subject in need thereof.

In some embodiments, the present disclosure includes to a method of improving neurological function in a mammal suffering from cerebral autosomal dominant arteriopathy with subcortical infarcts and leukoencephalopathy (CADASIL), including administering an effective amount, such as a therapeutically acceptable amount, of a composition including a granulocyte colony-stimulating factor (G-CSF) polypeptide in combination with a stem cell factor (SCF) polypeptide.

Embodiments of the present disclosure advantageously target decreased and/or deficient VEGF for developing new treatments to restrict CADASIL development and disease progression and provide reparative effects to subjects in need thereof. In embodiments, the present disclosure advantageously provides improvement in neurological function characterized by slowing the progress of CADASIL, increased angiogenesis, increased vascular cell proliferation, restored neurogenesis, increased density of axons, increased density of dendrites, increased density of synapses, restored blood vessels, improved spatial learning, improved memory, enhanced neurostructural regeneration, enhanced synaptogenesis and/or enhanced neurogenesis.

In some embodiments, stem cell factor (SCF) polypeptide or SCF suitable for use herein may be one or more of a naturally-occurring SCF (e.g. natural human-SCF) as well as non-naturally occurring (i.e., different from naturally occurring) polypeptides having amino acid sequences and glycosylation sufficiently duplicative of that of naturally-occurring stem cell factor to allow possession of a hematopoietic biological activity of naturally-occurring stem cell factor. In some embodiments, SCF may refer to recombinantly produced SCF, or fragments, analogs, variants, or derivatives thereof as reported, for example in U.S. Pat. Nos. 6,204,363; 6,207,417; 6,207,454; 6,207,802; 6,218,148; and 6,248,319. In embodiments, stem cell factor has the ability to stimulate growth of early hematopoietic progenitors which are capable of maturing to erythroid, megakaryocyte, granulocyte, lymphocyte, and macrophage cells. U.S. Pat. No. 8,404,653 further describes stem cell factor (SCF) polypeptide, and isoforms thereof suitable for use in accordance with the present disclosure.

In some embodiments, granulocyte colony-stimulating factor (G-CSF) polypeptide or G-CSF may refer to one or more naturally-occurring human and heterologous species G-CSF, recombinantly produced G-CSF that is the expression product consisting of either 174 or 177 amino acids, or fragments, analogs, variants, or derivatives thereof as reported, for example in Kuga et al., Biochem. Biophys. Res. Comm. 159:103-111, 1989; Lu et al., Arch. Biochem. Biophys. 268:81-92, 1989; U.S. Pat. Nos. 4,810,643; 4,904,584; 5,104,651; 5,214,132; 5,218,092; 5,362,853; 5,606,024; 5,824,778; 5,824,784; 6,017,876; 6,166,183; and 6,261,550; U.S. Pat. Appl. No. US 2003/0064922. Included are chemically modified G-CSFs, see, e.g., those reported in WO 9012874, EP 0 401384, and EP 0 335423. See also, WO 03006501; WO 03030821; WO 0151510; WO 9611953; WO 9521629; WO 9420069; WO 9315211; WO 9305169; JP 04164098; WO 9206116; WO 9204455; EP 0473268; EP 0 456200; WO 9111520; WO 9105798; WO 9006952; WO 8910932; WO 8905824; WO 9118911; and EP 0 370205. Also encompassed herein are all forms of G-CSF, such as ALBUGRANIN™ brand G-CSF, NEULASTA™ brand G-CSF, NEUPOGEN® brand G-CSF, and GRANOCYTE® brand G-CSF.

In embodiments, one or more SCFs and/or G-SCFs are combined in a pharmaceutical composition in an amount to form a therapeutically effective amount of active ingredient in a pharmaceutical composition. In embodiments, SCF and G-SCF may be present in pharmaceutically acceptable ratios such as 80:20, 60:40: 50:50, 40:60, or 20:80 weight percent of the total composition.

In embodiments, one or more VEGFs, SCFs and/or G-SCFs are combined in a pharmaceutical composition in an amount to form a therapeutically effective amount of active ingredient in a pharmaceutical composition. In embodiments, VEGF, SCF and G-SCF may be present in pharmaceutically acceptable ratios such as 80:10:10, 70:15:15, 60:20:20, 50:25:25, 40:30:30: 20:40:40, 20:20:40, 10:40:50 or 10:50:40 weight percent of the total composition.

### Definitions

As used in the present specification, the following words and phrases are generally intended to have the meanings as set forth below, except to the extent that the context in which they are used indicates otherwise.

As used herein, the singular forms “a”, “an”, and “the” include plural references unless the context clearly dictates otherwise. Thus, for example, references to “a compound” include the use of one or more compound(s). “A step” of a method means at least one step, and it could be one, two, three, four, five or even more method steps.

As used herein the terms “about,” “approximately,” and the like, when used in connection with a numerical variable, generally refers to the value of the variable and to all values of the variable that are within the experimental error (e.g., within the 95% confidence interval [CI 95%] for the mean) or within +10% of the indicated value, whichever is greater.

As used herein the terms “drug,” “drug substance,” “active pharmaceutical ingredient,” and the like, refer to a compound (e.g., one or more peptides in accordance with the present disclosure such as VEGF, SCF, and/or G-SCF) that may be used for treating a subject in need of treatment.

As used herein the term “cDNA” refers to a DNA molecule that can be prepared by reverse transcription from an RNA molecule obtained from a eukaryotic or prokaryotic cell, a virus, or from a sample solution. In embodiments, cDNA lacks introns or intron sequences that may be present in corresponding genomic DNA. In embodiments, cDNA may refer to a nucleotide sequence that corresponds to the nucleotide sequence of an RNA from which it is derived. In embodiments, cDNA refers to a double-stranded DNA that is complementary to and derived from mRNA.

As used herein the “degree of identity” refers to the relatedness between two amino acid sequences or between two nucleotide sequences and is described by the parameter “identity”. In embodiments, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid or nucleotide in the two aligned sequences on a given position in the alignment; and 3) dividing the number of exact matches with the length of the reference sequence. In one embodiment, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid; or nucleotide in the two aligned sequences on a given position in the alignment; and 3) dividing the number of exact matches with the length of the longest of the two sequences. In some embodiments, the degree of sequence identity refers to and may be calculated as described under “Degree of Identity” in U.S. Pat. No. 10,531,672 starting at Column 11, line 56. U.S. Pat. No. 10,531,672 is incorporated by reference in its entirety. In embodiments, an alignment program suitable for calculating percent identity performs a global alignment program, which optimizes the alignment over the full-length of the sequences. In embodiments, the global alignment program is based on the Needleman-Wunsch algorithm (Needleman, Saul B.; and Wunsch, Christian D. (1970), “A general method applicable to the search for similarities in the amino acid sequence of two proteins”, Journal of Molecular Biology 48 (3): 443-53). Examples of current programs performing global alignments using the Needleman-Wunsch algorithm are EMBOSS Needle and EMBOSS Stretcher programs, which are both available on the world wide web at www.ebi.ac.uk/Tools/psa/. In some embodiments a global alignment program uses the Needleman-Wunsch algorithm and the sequence identity is calculated by identifying the number of exact matches identified by the program divided by the “alignment length”, where the alignment length is the length of the entire alignment including gaps and overhanging parts of the sequences. In embodiments, the mafft alignment program is suitable for use herein.

As used herein the term “excipient” or “adjuvant” refers to any inert substance.

As used herein the terms “drug product,” “pharmaceutical dosage form,” “dosage form,” “final dosage form” and the like, refer to a pharmaceutical composition that is administered to a subject in need of treatment and generally may be in the form of tablets, capsules, sachets containing powder or granules, liquid solutions or suspensions, patches, and the like.

As used herein the term “pharmaceutically acceptable” substances refers to those substances which are within the scope of sound medical judgment suitable for use in contact with the tissues of subjects, such as e.g., substances without undue toxicity, irritation, allergic response, and the like, and effective for their intended use.

As used herein the term “pharmaceutical composition” refers to the combination of one or more drug substances such as e.g., one or more peptides in accordance with the present disclosure and one or more excipients and one or more pharmaceutically acceptable vehicles with which the one or more peptides in accordance with the present disclosure is administered to a subject.

As used herein, the term “pharmaceutically acceptable salt” refers to a salt of a compound, which possesses the desired pharmacological activity of the parent compound. Non-limiting examples of pharmaceutically acceptable salts include: acid addition salts, formed with inorganic acids such as hydrochloric acid, hydrobromic acid, sulfuric acid, nitric acid, phosphoric acid, and the like; or formed with organic acids; and salts formed when an acidic proton present in the parent compound is replaced by a metal ion, for example, an alkali metal ion, an alkaline earth ion, or an aluminum ion. Acetate salts are also a pharmaceutically acceptable salt for use herein.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

As used herein the term “pharmaceutically acceptable vehicle” refers to a diluent, adjuvant, excipient or carrier with which a compound is administered.

As used herein the term “prevent”, “preventing” and “prevention” of CADASIL disease means (1) reducing the risk of a patient who is not experiencing symptoms of CADASIL from developing CADISIL disease, or (2) reducing the frequency of, the severity of, or a complete elimination of CADASIL symptoms already being experienced by a subject.

The term “recombinant” when used herein to characterize a DNA sequence such as a plasmid, vector, or construct refers to an artificial combination of two otherwise separated segments of sequence, e.g., by chemical synthesis and/or by manipulation of isolated segments of nucleic acids by genetic engineering techniques.

As used herein the term “subject” includes humans, animals or mammals. The terms “subject” and “patient” may be used interchangeably herein.

The term “substantially purified,” as used herein, refers to a component of interest that may be substantially or essentially free of other components which normally accompany or interact with the component of interest prior to purification. By way of example only, a component of interest may be “substantially purified” when the preparation of the component of interest contains less than about 30%, less than about 25%, less than about 20%, less than about 15%, less than about 10%, less than about 5%, less than about 4%, less than about 3%, less than about 2%, or less than about 1% (by dry weight) of contaminating components. Thus, a “substantially purified” component of interest may have a purity level of about 70%, about 75%, about 80%, about 85%, about 90%, about 95%, about 96%, about 97%, about 98%, about 99% or greater.

The term “therapeutically effective amount” as used herein refers to an amount of an agent sufficient to achieve, in a single or multiple doses, the intended purpose of treatment. A “therapeutically effective amount” can vary depending, for example, on the compound, the severity of the disease, the age of the subject to be treated, comorbidities of the subject to be treated, existing health conditions of the subject, and/or the weight of the subject to be treated. A “therapeutically effective amount” is an amount sufficient to alter the subjects' natural state.

The term “treatment” as used herein refers to alleviation of one or more symptoms or features associated with the presence of the particular condition or suspected condition being treated. Treatment does not necessarily mean complete cure or remission, nor does it preclude recurrence or relapses. Treatment can be effected over a short term, over a medium term, or can be a long-term treatment, such as, within the context of a maintenance therapy. Treatment can be continuous or intermittent.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Before embodiments are further described, it is to be understood that this disclosure is not limited to particular embodiments described, as such may, of course, vary. It is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### DESCRIPTION OF CERTAIN EMBODIMENTS

The present disclosure includes compositions such as pharmaceutical compositions or drug products, and methods for preventing, treating, or ameliorating cerebral autosomal dominant arteriopathy with subcortical infarcts and leukoencephalopathy (CADASIL), including administering vascular endothelial growth factor (VEGF), or functional isoforms thereof, to a subject in need thereof. In some embodiments, methods for preventing, treating, or ameliorating cerebral autosomal dominant arteriopathy with subcortical infarcts and leukoencephalopathy (CADASIL), include administering vascular endothelial growth factor (VEGF), or isoforms thereof, to a brain of a subject in need thereof. In embodiments, the administering includes any suitable method of increasing the amount of VEGF in a subject in need thereof. For example, an initial amount of VEGF in the brain of a subject may be increased to a second amount, or predetermined second amount of VEGF in the brain of a subject. In embodiments, VEGF, or functional isoforms thereof, include one or more of VEGFa, VEGFb, VEDFc, or combinations thereof. In embodiments, the VEGF, or functional isoforms thereof, include or consist of an amino acid sequence having at least 90%, 95%, 97%, 99% sequence identity to SEQ ID NO: 1, SEQ ID NO: 2, SEQ ID NO:3, SEQ ID NO:4; SEQ ID NO:5, or SEQ ID NO:6, or a pharmaceutically acceptable salt of any thereof. In embodiments, administering VEGF, or functional isoforms thereof, includes administering one or more agents, compositions, or pharmaceutical compositions that increase VEGF in a subject, such as administering a granulocyte colony-stimulating factor (G-CSF) polypeptide, alone, or in combination with a stem cell factor (SCF) polypeptide, in an amount sufficient, such as a therapeutically acceptable amount, to increase VEGF in a subject in need thereof. In embodiments, administering VEGF, or functional isoforms thereof, includes administering: 1) a cDNA that encodes one or more proteins including or consisting of VEGFa, VEGFb, VEGFc, a VEGF isoform, or combinations thereof; or 2) one or more nucleic acid sequences that encode one or more proteins including or consisting of a VEGFa, VEGFb, VEGFc, a VEGF isoform, or combinations thereof. In embodiments, VEGF is provided in a pharmaceutical composition, in a therapeutically effective amount for the treatment of CADISIL. In embodiments, VEGF is in the form of a pharmaceutically acceptable salt, such as an acetate. In embodiments, the pharmaceutical composition comprises or consists of a pharmaceutically effective vehicle. In some embodiments, the composition includes recombinant excipients, or other excipients.

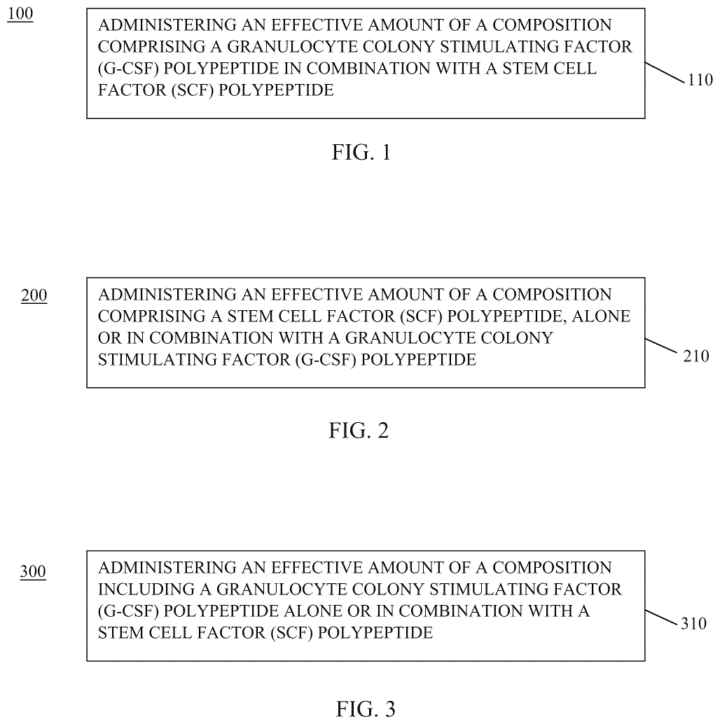

Referring now to FIG. 1, method 100 is shown relating to improving neurological function and cerebral blood flow and enhancing cerebrovascular maintenance and regeneration in a mammal suffering from cerebral autosomal dominant arteriopathy with subcortical infarcts and leukoencephalopathy (CADASIL). At process sequence 110, method 100 includes administering an effective amount, such as a therapeutically effective amount, of a composition including a granulocyte colony-stimulating factor (G-CSF) polypeptide in combination with a stem cell factor (SCF) polypeptide. In some embodiments, improvement in neurological function is characterized by improved cognitive function. In some embodiments, the improvement in neurological function is characterized by slowing the progress of CADASIL, increased angiogenesis, increased vascular cell proliferation, restored neurogenesis, increased density of axons, increased density of dendrites, increased density of synapses, restored blood vessels, improved spatial learning, improved memory, enhanced neurostructural regeneration, enhanced synaptogenesis and enhanced neurogenesis. In some embodiments, the mammal is a mouse or a human. In some embodiments, the administering further includes targeting VEGF with the composition in an amount sufficient to increase VEGF and/or VEGF-regulated angiogenesis. In some embodiments, the composition is characterized as pharmaceutically acceptable, or as a pharmaceutical composition. In some embodiments, the composition further includes a pharmaceutically acceptable salt, or is disposed within a pharmaceutically acceptable vehicle. In some embodiments, the composition is a drug product and includes recombinant excipients, or other excipients.

Referring now to FIG. 2, the present disclosure depicts method 200 relating to a method of improving neurological function and cerebral blood flow and enhancing cerebrovascular maintenance and regeneration in a mammal suffering from cerebral autosomal dominant arteriopathy with subcortical infarcts and leukoencephalopathy (CADASIL). At process sequence 210, method 200 incudes administering an effective amount, such as a therapeutically acceptable amount, of a composition including a stem cell factor (SCF) polypeptide, alone or in combination with a granulocyte colony-stimulating factor (G-CSF) polypeptide. In some embodiments, the composition includes a SCF polypeptide alone. In some embodiments, the improvement in neurological function is characterized by improved sensorimotor skills and coordination, or an increase in VEGF and/or VEGF-regulated angiogenesis. In some embodiments, the mammal is a mouse or a human. In some embodiments, the methods of the present disclosure restore neurogenesis and the densities of axons, dendrites, and synapses.

Referring now to FIG. 3, method 300 is shown relating to improving neurological function and cerebral blood flow and enhancing cerebrovascular maintenance and regeneration in a mammal suffering from cerebral autosomal dominant arteriopathy with subcortical infarcts and leukoencephalopathy (CADASIL), including administering an effective amount, such as a therapeutically effective amount of a composition including a granulocyte colony-stimulating factor (G-CSF) polypeptide alone or in combination with a stem cell factor (SCF) polypeptide. In embodiments, the composition includes a G-CSF polypeptide alone. In some embodiments, the improvement in neurological function is characterized by improved sensorimotor skills and coordination. In some embodiments, the mammal is a mouse or a human.

In embodiments, the present disclosure relates to a pharmaceutical composition for improving neurological function, cerebral blood flow, and/or enhancing cerebrovascular maintenance and regeneration in a subject in need thereof such as a mammal suffering from cerebral autosomal dominant arteriopathy with subcortical infarcts and leukoencephalopathy (CADASIL). In embodiments, the pharmaceutical composition is characterized as pharmaceutically acceptable. In embodiments, the pharmaceutical compositions include one or more granulocyte colony-stimulating factor (G-CSF) polypeptide of the present disclosure, or isoforms thereof, in combination with a stem cell factor (SCF) polypeptide of the present disclosure, or isoforms thereof. In embodiments, the pharmaceutical composition includes one or more physiologically compatible buffers, one or more pharmaceutically acceptable carriers or excipients. In embodiments the pharmaceutical compositions, wherein the composition for bolstering VEGF function or performance to treat or alleviate pathology and symptoms relating to CADASIL.

In embodiments, the present disclosure includes a pharmaceutical composition including one or more active pharmaceutical ingredients that increase or target VEGF in a subject in need thereof. Non-limiting examples of one or more active pharmaceutical ingredients include one or more of VEGF, or functional isoforms thereof, including one or more of VEGFa, VEGFb, VEDFc, or combinations thereof, or pharmaceutically acceptable salts thereof. In embodiments, the VEGF, or functional isoforms thereof, include or consist of an amino acid sequence having at least 90%, 95%, 97%, 99% sequence identity to SEQ ID NO: 1, SEQ ID NO: 2, SEQ ID NO:3, SEQ ID NO:4; SEQ ID NO:5, or SEQ ID NO:6 or a pharmaceutically acceptable salt thereof. In embodiments, the compositions may include one or more pharmaceutically acceptable agents or compositions that increase VEGF in a subject, such as a granulocyte colony-stimulating factor (G-CSF) polypeptide, alone, or in combination with a stem cell factor (SCF) polypeptide, or a pharmaceutically acceptable salt thereof, in an amount sufficient, such as a therapeutically acceptable amount, to increase VEGF in a subject in need thereof.

In some embodiments, the compositions of the present disclosure include a pharmaceutically acceptable carrier or diluent. In embodiments, the carrier(s) or diluent(s) are compatible with the other ingredients of the composition and not deleterious to the recipient thereof. Typically, carriers for injection, and the final composition, are sterile. Preparation of a composition of the present disclosure can be carried out using standard pharmaceutical preparation chemistries and methodologies all of which are readily available to the reasonably skilled artisan. For example, peptides or pharmaceutically acceptable salts thereof can be combined with one or more pharmaceutically acceptable excipients or vehicles. See e.g., U.S. Pat. No. 9,657,061 herein incorporated by reference.

In embodiments, auxiliary substances, such as wetting or emulsifying agents, tonicity agents, pH buffering substances and the like, may be present in the excipient or vehicle. In embodiments, excipients, vehicles and auxiliary substances are generally pharmaceutical agents which may be administered without undue toxicity. Pharmaceutically acceptable excipients include, but are not limited to, liquids such as water, saline, and alcohol. A thorough discussion of pharmaceutically acceptable excipients, vehicles and auxiliary substances is available in Remington's Pharmaceutical Sciences (Mack Pub. Co., N.J. 1991). In embodiments, pharmaceutical compositions may be prepared, packaged, or sold in a form suitable for bolus administration or for continuous administration.

In embodiments, injectable compositions may be prepared, packaged, or sold in unit dosage form, such as in ampoules or in multi-dose containers containing a preservative. In embodiments, pharmaceutical compositions include suspensions, solutions, emulsions in oily or aqueous vehicles, pastes. In embodiments, pharmaceutical compositions may include one or more additional ingredients including suspending, stabilizing, or dispersing agents. In embodiments, pharmaceutical compositions may be prepared, packaged, or sold in the form of a sterile injectable aqueous or oily suspension or solution. In embodiments, suspension or solution may be prepared according to the known art. Other suitable compositions and excipients suitable for use herein are described in U.S. Pat. No. 9,657,061, herein entirely incorporated by reference.

The preparation of any of the peptides or pharmaceutically acceptable salts thereof mentioned herein will depend upon factors such as the nature of the substance and the method of delivery. Any such substance may be administered in a variety of dosage forms. It may be administered orally (e.g. as tablets, troches, lozenges, aqueous or oily suspensions, dispersible powders or granules), parenterally, subcutaneously, by inhalation, intradermally, intravenously, intramuscularly, intrasternally, transdermally or by infusion techniques. In embodiments, inhalation through the nose is a suitable route for administration. A physician will be able to determine the required route of administration for each particular individual.

In embodiments, the compositions of the present disclosure include a suitable concentration of each peptide or salt to be effective without causing adverse reaction. In embodiments, the concentration of each peptide or salt in the composition will be in the range of 0.03 to 400 nmol/ml.

In embodiments, administering VEGF, or functional isoforms thereof, includes administering: 1) a cDNA that encodes one or more proteins including or consisting of VEGFa, VEGFb, VEGFc, a VEGF isoform, or combinations thereof; or 2) one or more nucleic acid sequences that encode one or more proteins including or consisting of a VEGFa, VEGFb, VEGFc, a VEGF isoform, or combinations thereof. In embodiments, suitable cDNA or nucleic acid sequences for administration include those that encode the peptides in SEQ ID NOS: 1-6, and highly related sequences such as those having at least 90%, 95%, 97%, or 99% sequence identity thereto.

In embodiments, nucleic acid sequences that encode one or more VEGF proteins or proteins that increase the amount of VEGF in a subject in need thereof may undergo procedures to allow for expression of the nucleic acid sequences in a host cell. Suitable mammalian expression systems, vectors, cell delivery systems, and methods of treatment, and administration of nucleic acid vectors, suitable for use with the nucleic acids of the present disclosure, are described in U.S. Patent Publication No. 20180110879, herein incorporated by reference in its entirety.

In embodiments, administering VEGF, or functional isoforms thereof, includes administering: 1) a cDNA that encodes one or more proteins including or consisting of VEGFa, VEGFb, VEGFc, a VEGF isoform, or combinations thereof; or 2) one or more nucleic acid sequences that encode one or more proteins including or consisting of a VEGFa, VEGFb, VEGFc, a VEGF isoform, or combinations thereof.

In embodiments, administering one or more SCFs, or functional isoforms thereof, includes administering: 1) a cDNA that encodes one or more proteins including or consisting of an SCF, or combinations thereof; or 2) one or more nucleic acid sequences that encode one or more proteins including or consisting of an SCF, an SCF isoform, or combinations thereof.

In embodiments, administering one or more G-SCFs, or functional isoforms thereof, includes administering: 1) a cDNA that encodes one or more proteins including or consisting of a G-SCF; or 2) one or more nucleic acid sequences that encode one or more proteins including or consisting of a G-SCF, a G-SCF isoform, or combinations thereof.

In embodiments, administering one or more SCFs and/or G-SCFs, or functional isoforms thereof, includes administering: 1) a cDNA that encodes one or more proteins including or consisting of an SCF and/or G-SCF, or combinations thereof; or 2) one or more nucleic acid sequences that encode one or more proteins including or consisting of a SCF and/or G-SCF, isoforms thereof, or combinations thereof.

In embodiments, administering one or more VEGFs, SCFs and/or G-SCFs, or functional isoforms thereof, includes administering: 1) a cDNA that encodes one or more proteins including or consisting of VEGF, SCF and/or G-SCF, or combinations thereof; or 2) one or more nucleic acid sequences that encode one or more proteins including or consisting of a VEGF, SCF and/or G-SCF, isoforms thereof, or combinations thereof.

Methods of administering nucleic acids, or cDNA's to obtain the benefit of proteins or protein fragments expressed therefrom are known in the art and are suitable for use herein. See e.g., suitable mammalian expression systems, vectors, cell delivery systems, methods of treatment, and administration of nucleic acid vectors, that are suitable for use with the nucleic acids of the present disclosure, as described in U.S. Patent Publication No. 20180110879, herein incorporated by reference in its entirety.

In some embodiments the present disclosure provides a method for treating or ameliorating cerebral autosomal dominant arteriopathy with subcortical infarcts and leukoencephalopathy (CADASIL), including administering vascular endothelial growth factor (VEGF), or functional isoforms thereof, to a brain of a subject in need thereof. In embodiments, VEGF, or functional isoforms thereof, comprise one or more of VEGFa, VEGFb, VEDFc, or combinations thereof. In embodiments, the VEGF, or functional isoforms thereof, comprise an amino acid sequence having at least 90%, 95%, 97% or 99% sequence identity to SEQ ID NO: 1, SEQ ID NO: 2, SEQ ID NO:3, SEQ ID NO:4; SEQ ID NO:5, or SEQ ID NO:6. In embodiments, administering VEGF, or functional isoforms thereof, comprises administering a granulocyte colony-stimulating factor (G-CSF) polypeptide in combination with a stem cell factor (SCF) polypeptide, in an amount sufficient to increase VEGF in a subject in need thereof. In embodiments, administering VEGF, or functional isoforms thereof, comprises administering: 1) a cDNA that encodes one or more proteins including VEGFa, VEGFb, VEGFc, a VEGF isoform, or combinations thereof; or 2) one or more nucleic acid sequences that encode one or more proteins including a VEGFa, VEGFb, VEGFc, a VEGF isoform, or combinations thereof.

In some embodiments, the present disclosure provides a method of improving neurological function and enhancing cerebrovascular maintenance and regeneration in a mammal suffering from cerebral autosomal dominant arteriopathy with subcortical infarcts and leukoencephalopathy (CADASIL), including: administering an effective amount of a pharmaceutically acceptable vascular endothelial growth factor (VEGF), or a functional isoform thereof, to a subject in need thereof. In embodiments, a pharmaceutically acceptable composition including a granulocyte colony-stimulating factor (G-CSF) polypeptide is administered in combination with a stem cell factor (SCF) polypeptide, in an amount effective in increasing VEGF in a subject in need thereof. In embodiments, an improvement in neurological function is characterized by improved cognitive function. In embodiments, an improvement in neurological function and an enhancement of cerebrovascular maintenance and regeneration is characterized by slowing a progress of CADASIL, increased blood vessel density, increased angiogenesis, increased vascular cell proliferation, restored cerebral blood vessels, restored neurogenesis, increased density of axons, increased density of dendrites, increased density of synapses, improved spatial learning, improved memory, enhanced neurostructural regeneration, enhanced synaptogenesis and enhanced neurogenesis. In embodiments, the subject is a human or a mouse. In embodiments, administering further comprises targeting VEGF with the composition in an amount sufficient to increase VEGF and/or VEGF-regulated angiogenesis and cell survival signaling. In embodiments, VEGF is increased at least 5×, 10×, 20× compared to an otherwise untreated subject.

In embodiments, the present disclosure includes a method of improving neurological function and cerebral blood flow and enhancing cerebrovascular maintenance and regeneration in a mammal suffering from cerebral autosomal dominant arteriopathy with subcortical infarcts and leukoencephalopathy (CADASIL), including administering an effective amount of a composition including a stem cell factor (SCF) polypeptide, alone or in combination with a granulocyte colony stimulating factor (G-CSF) polypeptide. In embodiments, the composition comprises a SCF polypeptide alone. In embodiments, an improvement in neurological function is characterized by improved sensorimotor skills and coordination and ameliorated depression and anxiety. In embodiments, an effective amount or therapeutically effective amount, is an amount sufficient to increase a VEGF and/or a VEGF-regulated angiogenesis and cell survival signaling. In embodiments, restored neurogenesis includes restoration of a density of axons, dendrites, and/or synapses.

In embodiments, a method of improving neurological function and cerebral blood flow and enhancing cerebrovascular maintenance and regeneration in a mammal suffering from cerebral autosomal dominant arteriopathy with subcortical infarcts and leukoencephalopathy (CADASIL), including administering an effective amount of a composition including a granulocyte colony-stimulating factor (G-CSF) polypeptide alone or in combination with a stem cell factor (SCF) polypeptide. In embodiments, an improvement in neurological function is characterized by improved sensorimotor skills and coordination and ameliorated depression and anxiety. In embodiments, an increase of VEGF and/or VEGF-regulated angiogenesis and cell survival signaling. In embodiments, the mammal is a mouse or a human. In embodiments, the method restores neurogenesis and a density of axons, dendrites, and synapses.

### Examples

### Introduction to Example Section

The present disclosure demonstrates that defective vascular endothelial growth factor (VEGF) in mural cells plays an essential role in the development of CADASIL. Defective VEGF is a novel therapeutic target for developing new treatments to restrict CADASIL development and disease progression. The working examples below demonstrate that decreased VEGF production and decreased VEGF-related cell signaling activation were seen in the mural cells of TgNotch3R90C mice at about 3 months of age, which is about 7 months earlier than VSMC degeneration and cerebrovascular dysfunction that happen at the age of 10 months in TgNotch3R90C mice. Treatments targeting amelioration of the defective VEGF have reparative effects in TgNotch3R90C mice.

Stem cell factor (SCF) and granulocyte-colony stimulating factor (G-CSF) are the essential hematopoietic growth factors and play key roles in regulating blood cell production and bone marrow cell survival and mobilization (See e.g., Welte, K., E. Platzer, et al. (1985). “Purification and biochemical characterization of human pluripotent hematopoietic colony-stimulating factor.” Proc Natl Acad Sci USA 82(5): 1526-1530; and Zsebo, K. M., J. Wypych, et al. (1990). “Identification, purification, and biological characterization of hematopoietic stem cell factor from buffalo rat liver-conditioned medium.” Cell 63(1): 195-201). SCF+G-CSF has been shown to have synergistic effects in enhancing proliferation, differentiation, survival and mobilization of hematopoietic stem cells (See e.g., Duarte R F, Frank D A (2000) SCF and G-CSF lead to the synergistic induction of proliferation and gene expression through complementary signaling pathways. Blood 96 (10); Duarte R F, Frank D A (2002) The synergy between stem cell factor (SCF) and granulocyte colony-stimulating factor (G-CSF): molecular basis and clinical relevance. Leukemia & lymphoma 43 (6): 1179-1187; and Hess D A, et al., (2002) Functional analysis of human hematopoietic repopulating cells mobilized with granulocyte colony stimulating factor alone versus granulocyte colony-stimulating factor in combination with stem cell factor. Blood 100 (3)). Earlier studies have also revealed the synergistic efficacy of SCF+G-CSF in promoting neurite outgrowths (See e.g., Su Y, Cui L, Piao C, Li B, Zhao L R (2013) The effects of hematopoietic growth factors on neurite outgrowth. PLOS One 8 (10)), and in enhancing brain repair in experimental chronic stroke. Moreover, it is now demonstrated that repeated treatments of SCF+G-CSF beginning at 9 months of age (one month earlier than VSMC degeneration occurring at the age of 10 months) in a transgenic mouse model of CADASIL (TgNotch3R90C mice) improves cognitive function, reduces VSMC degeneration, increases cerebrovascular density, and decreases capillary thrombosis observed at the age of 22 months (See e.g., Liu X Y, et al., (2015) Stem cell factor and granulocyte colony-stimulating factor exhibit therapeutic effects in a mouse model of CADASIL. Neurobiol Dis 73:189-203; and Ping S, Qiu X, Gonzalez-Toledo M E, Liu X, Zhao L R (2018) Stem Cell Factor in Combination with Granulocyte Colony-Stimulating Factor reduces Cerebral Capillary Thrombosis in a Mouse Model of CADASIL. Cell Transplant 27 (4): 637 647). However, how SCF+G-CSF treatment restricts progressive vascular pathology in CADASIL condition remains unknown.

Using brain sections from CADASIL patients, it was confirmed that blood vessel density in the brains of CADASIL patients decreased. It was thought that NOTCH3 mutation-caused blood vessel degeneration in the brain is the key pathological mechanism in CADASIL and that SCF+G-CSF-enhanced brain repair and cognitive recovery may be modulated through increasing blood vessel regeneration (i.e. angiogenesis). An angiogenic inhibitor was used to block the SCF+G-CSF-enhanced angiogenesis. This inhibitor is called bevacizumab (AVASTIN®) (Roche) which is an FDA-approved antibody therapy to inhibit angiogenesis through neutralizing VEGF-A. VEGF-A is also known as VEGF, which has been proven to be the most potent proangiogenic factor for promoting angiogenesis (See e.g., Vallon, M., J. Chang, et al. (2014). “Developmental and pathological angiogenesis in the central nervous system.” Cell Mol Life Sci 71(18): 3489-3506). During carrying out the experiments, it was discovered that VEGF plays a key role in pathogenesis of CADASIL and in mediating the SCF+G-CSF-enhanced brain repair and cognitive recovery in CADASIL mice (TgNotch3R90C mice) (See e.g., Ping, S., et al., (2019). “Stem cell factor and granulocyte colony-stimulating factor promote brain repair and improve cognitive function through VEGF-A in a mouse model of CADASIL.” Neurobiol Dis 132: 104561. To block the SCF+G-CSF-enhanced angiogenesis, Bevacizumab (AVASTIN®) was injected before administering SCF+G-CSF in CADASIL mice (TgNotch3R90C mice). It was observed that pretreatment with bevacizumab (AVASTIN®), an angiogenic inhibitor which neutralizes VEGF-A, completely eliminated the SCF+G-CSF-enhanced cerebrovascular density, cognitive function recovery, vascular and neuronal structure regeneration, synaptogenesis and neurogenesis in TgNotch3R90C mice. It was also confirmed that SCF+G-CSF-enhanced endothelial cell (EC) proliferation and angiogenesis in TgNotch3R90C mouse brain-isolated ECs were also blocked by bevacizumab (AVASTIN®) pretreatment. To further validate the effects of SCF+G-CSF in increasing VEGF production in the brains of TgNotch3R90C mice, brain samples were collected 24 hours after final injections of SCF+G CSF treatment. Surprisingly, a significant increase of VEGF protein levels was seen in TgNotch3R90C mice treated with SCF+G-CSF. Importantly, it was also discovered that there is a significant decrease of VEGF protein levels in the brains of TgNotch3R90C mice as compared to wild type (WT) control mice (See e.g., Ping, S., et al., (2019). “Stem cell factor and granulocyte colony-stimulating factor promote brain repair and improve cognitive function through VEGF-A in a mouse model of CADASIL.” Neurobiol Dis 132: 104561). These data suggest that SCF+G-CSF treatment repairs Notch3R90C mutation-damaged brain through the VEGF-A-mediated angiogenesis, which sheds new light on the mechanism underlying the SCF+G-CSF-enhanced brain repair in CADASIL. Importantly, this study provides novel insight into the involvement of VEGF in the pathogenesis of CADASIL and offers a novel molecular target to develop new treatments for CADASIL. It was discovered that in the brains of 3-month-old TgNotch3R90C mice, VEGF expression was significantly decreased in VSMCs of small vessels and in pericytes of capillaries. The results of in vitro studies were confirmed that VSMCs and pericytes isolated from the brains of 3-months-old TgNotch3R90C mice (Tg-VSMCs, Tg-pericytes) show significant decreases of VEGF and VEGFR2 activation. Knocking down VEGF in Tg-VSMCs leads to increases of VSMC death and elimination of SCF+G-CSF treatment-enhanced VSMC survival rate. In addition, VEGF treatment increases Tg-VSMC survival. It was also discovered that cell signaling activation that particularly modulates cell survival and metabolism (e.g., PI3K/AKT, MEK/ERK, NF-kB and P38) was also decreased in VSMCs of TgNotch3R90C mice. VEGF treatment as well as SCF+G CSF treatment enhanced the cell survival as well as restored cell survival and metabolic signaling activation in the brain-isolated VSMCs of TgNotch3R90C mice. These discoveries confirm that decreased VEGF in VSMCs and pericytes of TgNotch3R90C mice is tightly linked to pathogenesis of CADASIL. Administering VEGF can restrict Notch3 mutation-induced VSMC loss, and VEGF is required for SCF+G-CSF treatment-enhanced VSMC survival. These novel findings validate that VEGF plays a key role in pathogenesis of CADASIL and offers a new target for developing new treatments to restrict pathological progression of CADASIL.

In total, the discoveries of the present disclosure will have a high impact in CADASIL and move the CADASIL research field forward by identifying defective VEGF as a critical molecular mechanism of disease development and by demonstrating defective VEGF as a novel therapeutic target to development new treatment for CADASIL.

Animals and experimental design. Aspects of this example are fully detailed in Stem Cell Factor and Granulocyte Colony-stimulating Factor Promote Brain Repair and Improve Cognitive Function Through VEGF-A in A Mouse Model of CADASIL to Ping et al., Neurobiology of Disease 132 (2019) 104561 (herein incorporated entirely by reference). Transgenic mice carrying a full-length human NOTCH3 gene with the Arginine-to-Cysteine (Arg90Cys) mutation at amino acid position 90 driven by the SM22α promoter in mural cells were used as a mouse model of CADASIL (TgNotch3R90C). The original breeders were generously provided as gifts from Dr. Anne Joutel (Faculté de Médecine, Paris, France). Nine-month-old male TgNotch3R90C mice were randomly divided into four groups (n=11-17/group): a vehicle control group, a bevacizumab (AVASTIN®) treatment group, an SCF+G-CSF treatment group, and a group treated with both bevacizumab (AVASTIN®) and SCF+G-CSF. Age-matched wild type (WT) mice were used as WT controls. The first treatment was performed at 9 months of age. Recombinant mouse SCF (PeproTech) and recombinant human G-CSF (Amgen) (SCF: 200 μg/kg, diluted in saline; G-CSF: 50 μg/kg, diluted in 5% dextrose) or an equal volume of vehicle solution (50% of saline and 50% of 5% dextrose) was subcutaneously injected for 7 consecutive days. To block the VEGF-A-mediated angiogenesis, bevacizumab (AVASTIN®) (Roche) (15 mg/kg, i.p.) (anti-VEGF monoclonal antibody) was administered 1 h before SCF+G-CSF treatment. The same treatment was repeated again at the age of 10 months. Twenty-four hours after the final treatment at 10 months of age, three mice were randomly chosen from each group to assess the levels of VEGF in the brain through Western Blot. Seven weeks after completion of the second treatment paradigm (equal to 12 months of age), water maze test was performed in the remaining mice (n=8-14/group) to evaluate spatial learning and memory. At the age of 15 months, mice were euthanized to examine structural changes in the brain by immunohistochemistry.

Major findings. In Western Blot, it was found that VEGF/VEGF-A protein in the brains of TgNotch3R90C mice was decreased as compared to WT mice. SCF+G-CSF-treated TgNotch3R90C mice showed increases of VEGF/VEGF-A protein in the brain.

In water maze test, impaired spatial learning and memory was seen in TgNotch3R90C mice. SCF+G-CSF-improved spatial learning and memory in TgNotch3R90C mice was eliminated by Avastin pretreatment.

In immunohistochemistry and histochemistry analyses, it was observed that blood vessel density was reduced in the cortex, striatum and hippocampus of TgNotch3R90C mice. SCF+G-CSF treatment restored blood vessels in the cortex, striatum and hippocampus of TgNotch3R90C mice. Bevacizumab (AVASTIN®) pretreatment completely blocked the SCF+G-CSF-enhanced angiogenesis in the brains of TgNotch3R90C mice.

Using primary culture of endothelial cells (ECs) isolated from the brains of TgNotch3R90C mice, it was further confirmed that (1) decreased cell proliferation and tube formation (angiogenesis) were seen in the ECs isolated from the brains of TgNotch3R90C mice; (2) SCF+G-CSF treatment restored the ability of cell proliferation and tube formation in the ECs isolated from the brains of TgNotch3R90C mice; and (3) the SCF+G-CSF-enhanced EC proliferation and tube formation was completely eliminated by Avastin pretreatment.

In addition, reduced densities of axons and dendrites as well as decreased synaptic density in the cortex and hippocampus were found in TgNotch3R90C mice. Decreased neurogenesis was also seen in the neurogenic regions of TgNotch3R90C mice. SCF+G-CSF treatment restored neurogenesis and the densities of axons, dendrites, and synapses in the brains of TgNotch3R90C mice. Bevacizumab (AVASTIN®) pretreatment completely blocked the SCF+G-CSF-enhanced neurostructural regeneration, synaptogenesis and neurogenesis in the brains of TgNotch3R90C mice.

Importantly, through correlation analysis, it was observed a significantly positive correlation between blood vessel density and the densities of axons, dendrites, synapses and neurogenesis, and a significantly negative correlation between escape latency in water maze testing (the longer the escape latency is, the worse the cognitive function shows) and the densities of blood vessels, axons, dendrites, and synapses.

Altogether, the findings suggest that decreased levels of VEGF in the brains of TgNotch3R90C mice are linked to decreased blood vessel density, leading to loss of axons, dendrites, synapses and neurogenesis as well as impaired spatial learning and memory.

Impaired angiogenesis is further confirmed in cultured cerebral ECs of TgNotch3R90C mice. SCF+G-CSF-enhanced angiogenesis in ECs isolated from the brains of TgNotch3R90C mice is blocked by antibody against VEGF-A (AVASTIN).

SCF+G-CSF treatment-improved spatial learning and memory and SCF+G-CSF treatment-enhanced neurostructural regeneration, synaptogenesis and neurogenesis in the brains of TgNotch3R90C mice are eliminated by bevacizumab (AVASTIN®) pretreatment, indicating that SCF+G-CSF-enhanced brain repair in TgNotch3R90C mice is dependent on VEGF-regulated angiogenesis.

This study has demonstrated that VEGF deficiency plays an important role in the development and progression of CADASIL, and that increasing VEGF and VEGF-regulated angiogenesis is a key mechanism underlying the SCF+G-CSF-enhanced brain repair in TgNotch3R90C mice. The findings of this study have also revealed that VEGF is a critical and novel target for developing treatment to restrict the progression of CADASIL.

### Example II

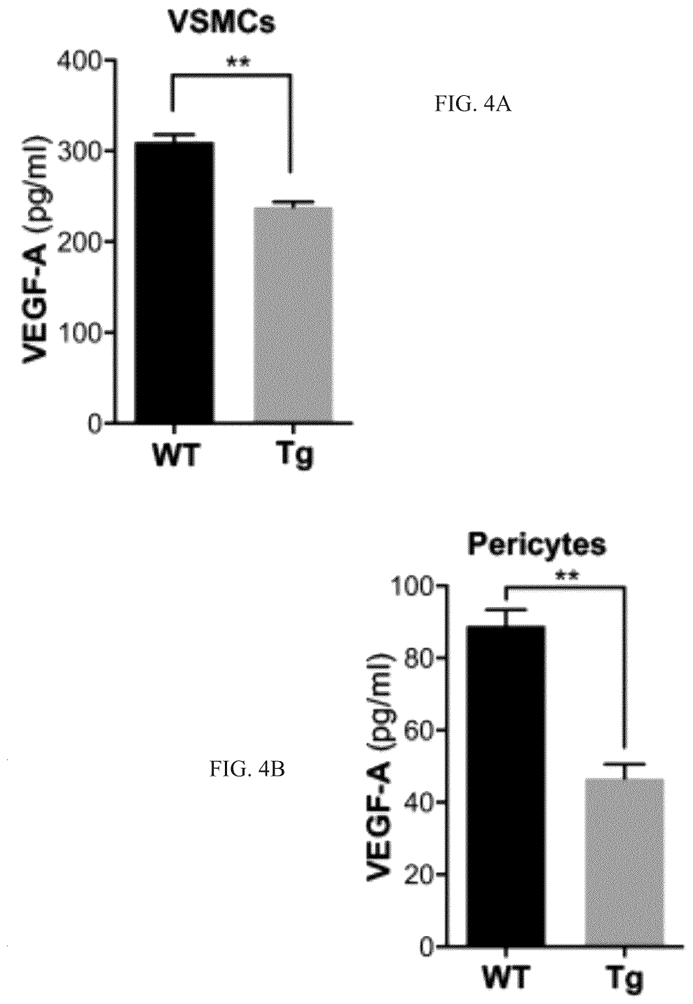

Decreased VEGF/VEGFR2 in cerebral mural cells of TgNotch3R90C mice. VSMCs and pericytes are the mural cells embedded within the basal lamina of blood vessels. VSMCs and pericytes were isolated from cerebral small vessels and cerebral capillaries, respectively, in 3-month-old TgNotch3R90C mice, cultured in cell culture dishes and used for experiments within 3 passages. Using an enzyme-linked immunosorbent assay, it was discovered that the levels of VEGF-A secreted from Tg-VSMCs and Tg-pericytes isolated from the brains of TgNotch3R90C mice were decreased as compared to WT-VSMCs and WT-pericytes isolated from the brains of age-matched WT mice (FIGS. 4A and 4B). FIGS. 4A and 4B are histograms showing secreted VEGF-A in VSMCs and pericytes by ELISA, respectively. Data from passage 1 cells, isolated from 3-month-old mouse brain. **p<0.01, repeated from three times.

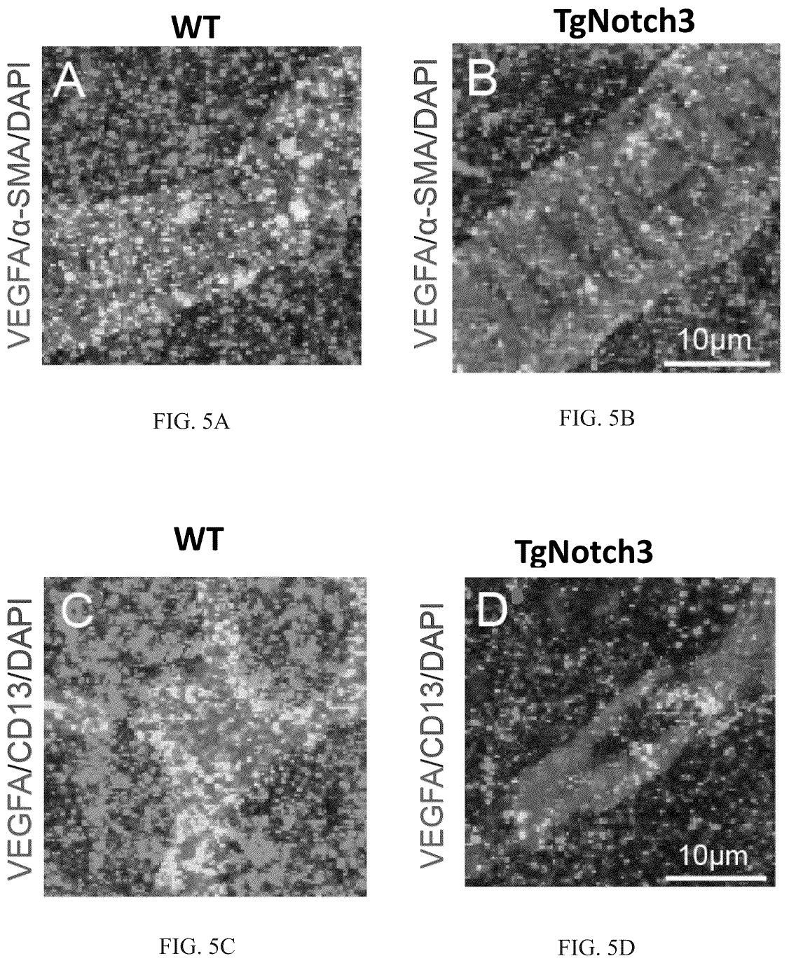

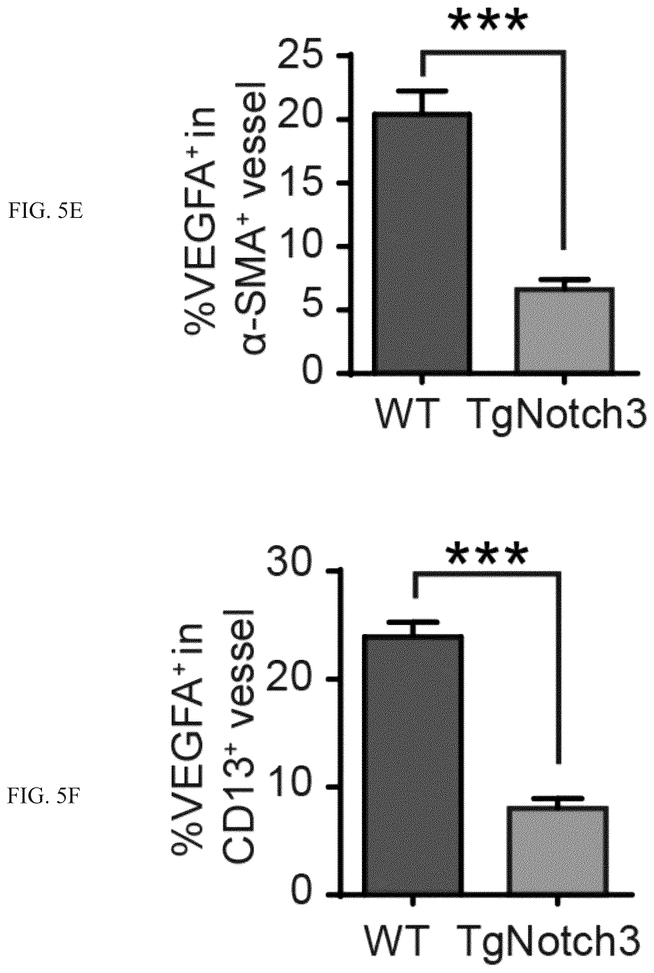

More specifically, FIGS. 5A-5F depicts immunohistochemistry data revealing the decreased VEGF expression (green) in both vascular smooth muscle cells (red, alpha. SMA positive cells) and pericytes (red, CD13 positive cells) in the brains of 3-month-old TgNotch3R90C mice. FIGS. 5A-D depict representative confocal images. FIGS. 5E-5F depict quantification data. ***p<0.001. WT: age matched wild type mice.

In addition, using immunohistochemistry in brain sections of 3-month-old TgNotch3R90C mice, it was discovered that VEGF expression levels were significantly decreased in both vascular smooth muscle cells (VSMCs) (alpha-SMA positive cells) of small blood vessels and pericytes (CD13 positive cells) of capillaries (FIGS. 5A-5F).

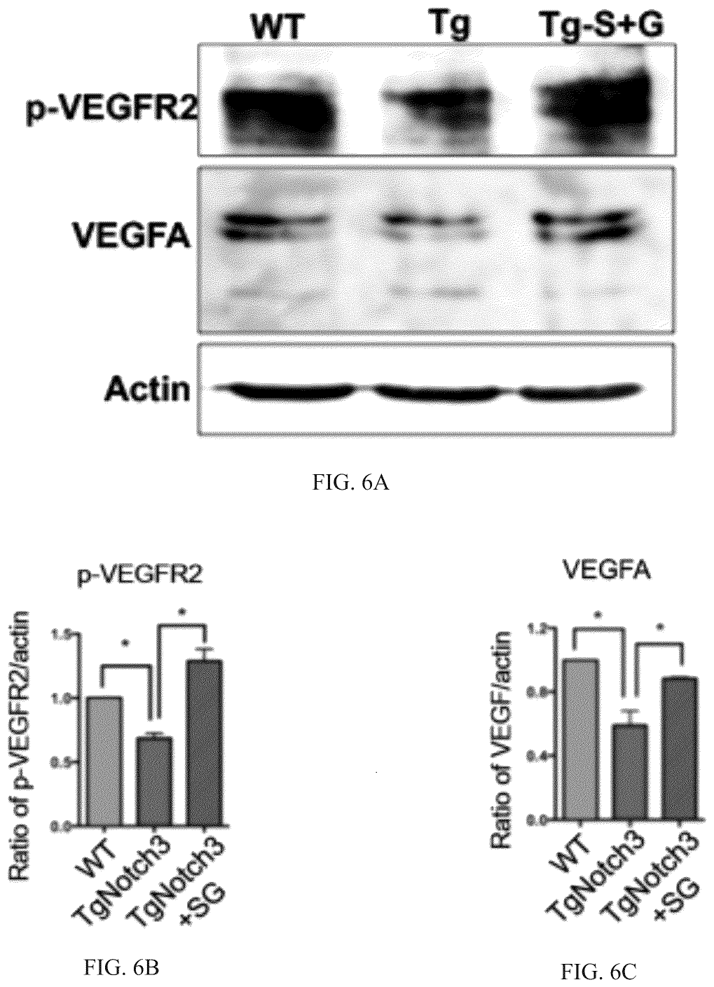

VEGFR2 is the major receptor of VEGF-A. It was also discovered that VEGF-A and phosphorylated VEGFR2 were decreased in Tg-VSMCs isolated from the brains of 3-month-old TgNotch3R90C mice. SCF+G-CSF treatment (20 ng/ml) elevated and restored VEGF-A and phosphorylated VEGFR2 in the Tg-VSMCs (FIGS. 6A-6C). FIGS. 6A, 6B, and 6C depict Western blot results and two histograms showing changes of VEGF-A and its receptor, VEGFR2. These findings demonstrate that decreased VEGF-A and decreased VEGFR2 activation are the important molecular pathology in cerebral mural cells with CADASIL-related Notch3 mutation. This unique molecular pathology of CADASIL is ameliorated by SCF+G-CSF treatment.

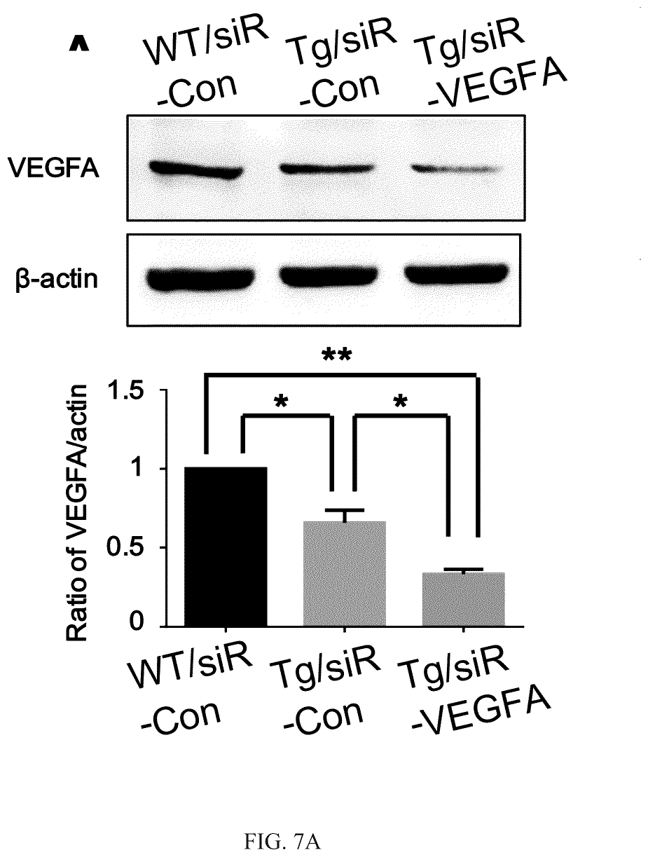

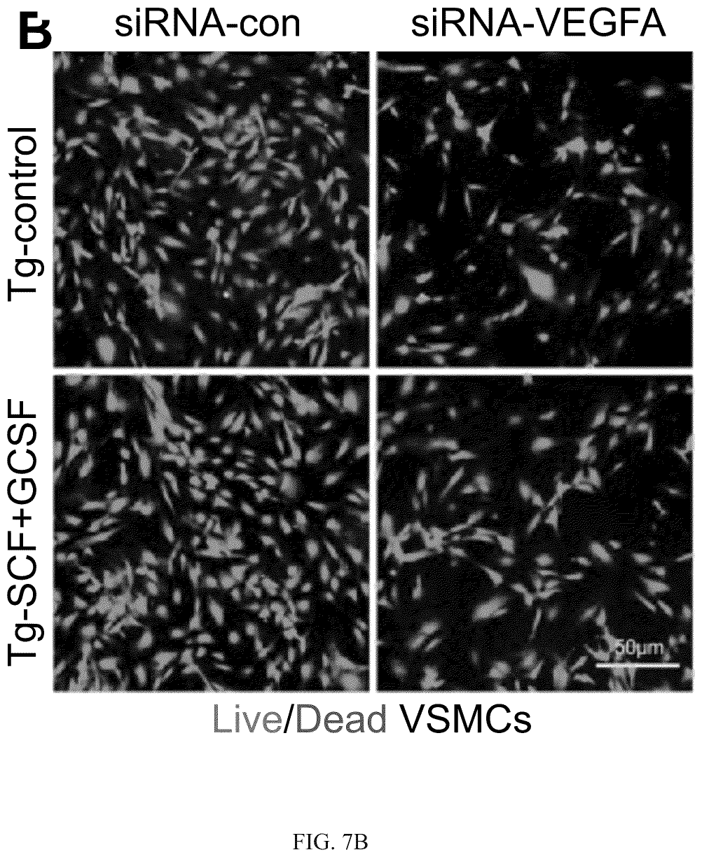

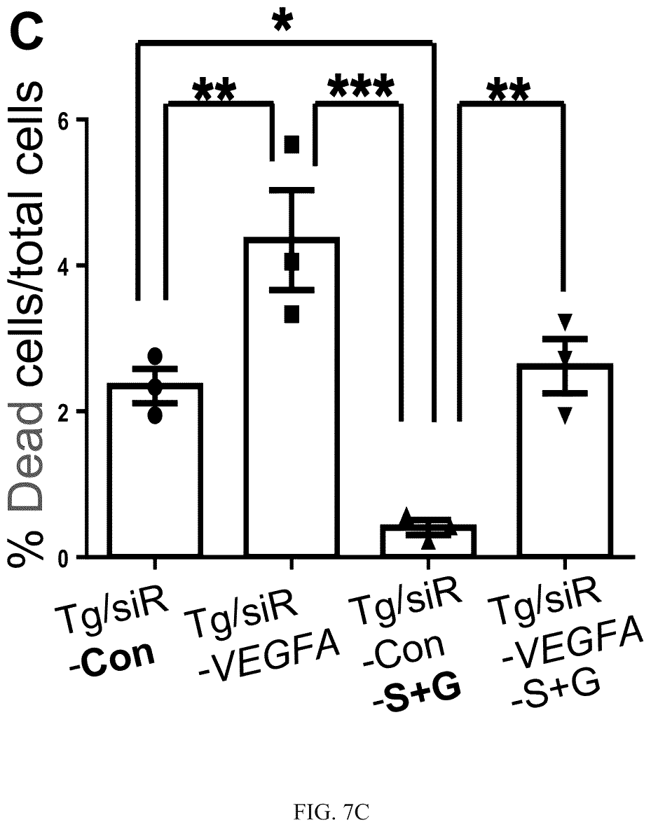

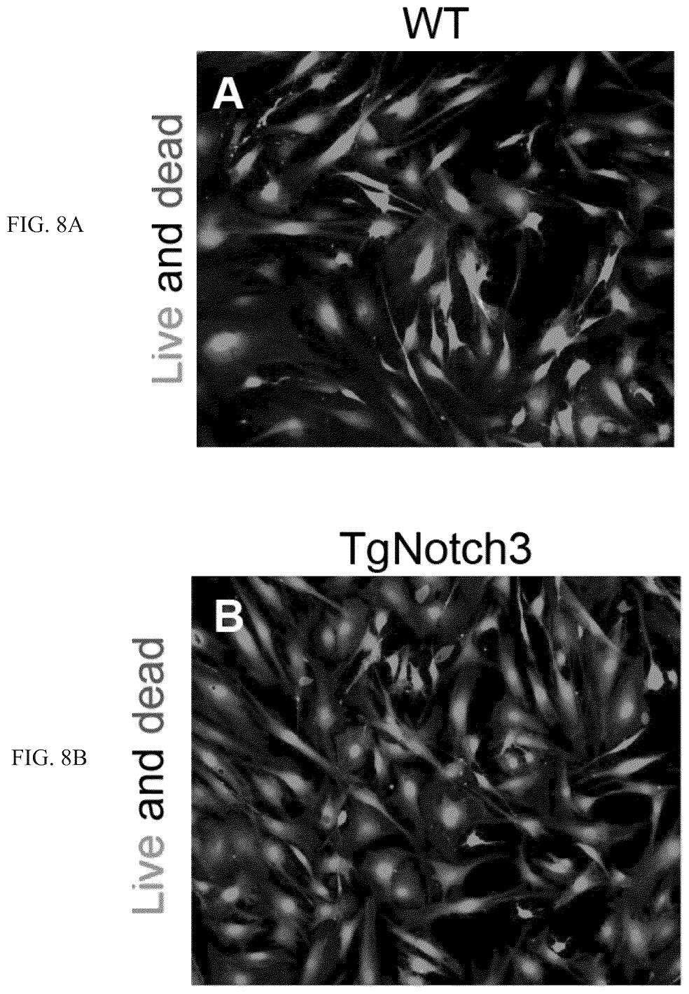

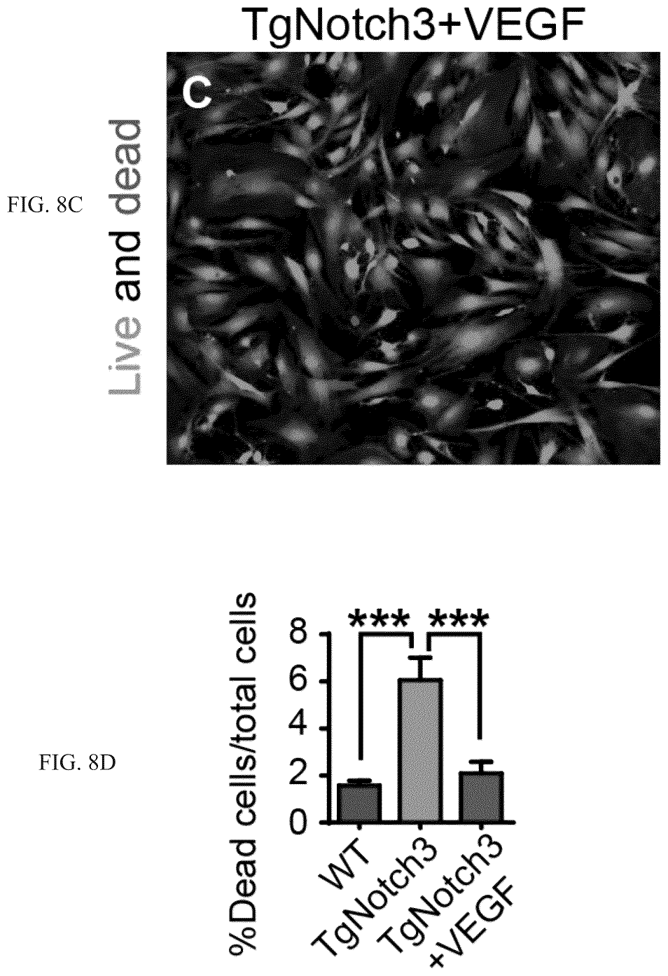

The role of VEGF in supporting mural cell survival in CADASIL condition. Using the approach of siRNAs to knock down VEGF-A in Tg-VSMCs isolated from the brains of 3-month-old TgNotch3R90C mice, it was uncovered that a lack of VEGF-A in Tg-VSMCs led to increased cell death in Tg-VSMCs. Knocking down VEGF-A in Tg-VSMCs using its siRNA resulted in elimination of SCF+G-CSF treatment-enhanced cell survival in Tg-VSMCs (FIGS. 7A-7C). This discovery reveals that VEGF is required for VSMC (vascular smooth muscle cells) survival and SCF+G-CSF treatment-enhanced VSMC survival in CADASIL condition. Moreover, additional discovery was revealed from the findings showing that cell death rate was significantly increased in the VSMCs isolated from the brains of 3-month-old TgNotch3R90C mice, and that providing VEGF treatment to these Tg-VSMCs led to robust reductions of cell death (FIGS. 8A-8D). This discovery further confirms that deficient VEGF in cerebral mural cells plays a key role in driving cerebral mural cell degeneration in the context of CADASIL, and that VEGF treatment has potential therapeutic value for ameliorating CADASIL-caused cerebral mural cell degeneration.

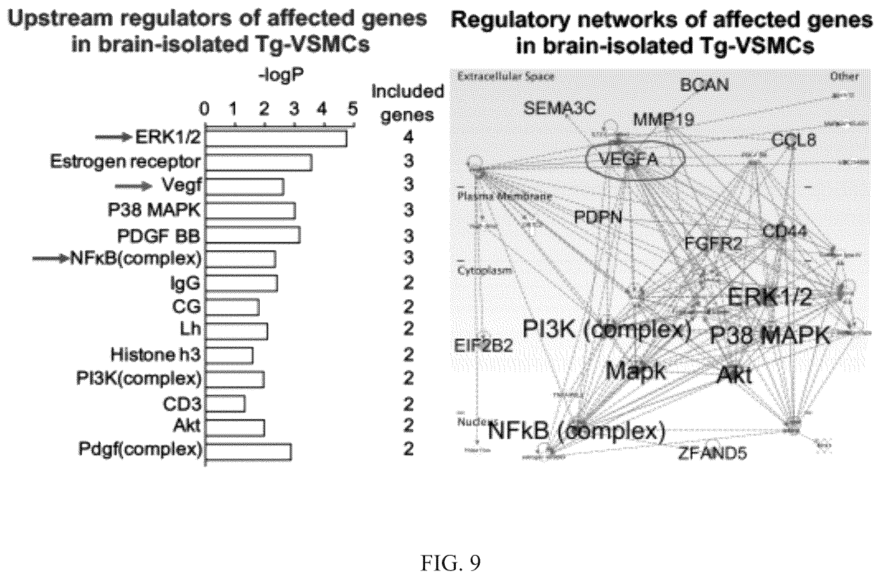

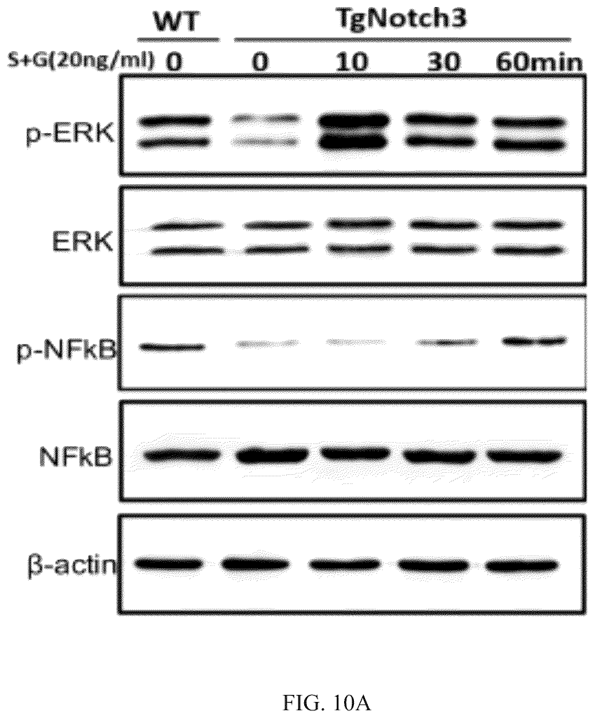

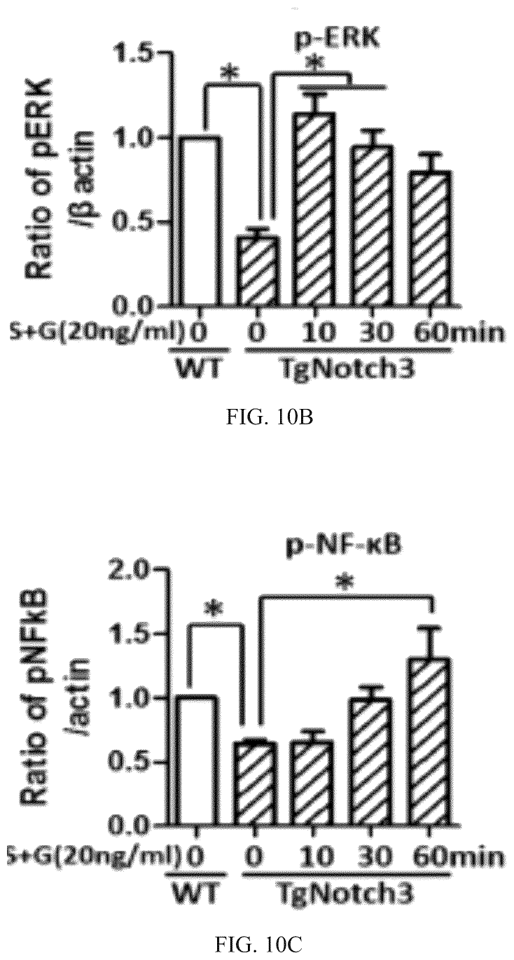

Defective VEGF and VEGF-related cell signaling in mural cells of TgNotch3R90C mice. Using a comprehensive, unbiased, reliable and sensitive RNA sequencing approach (NextSeq 500/500 High Output, Illumina), further analyzing the transcriptomic profiling of the VSMCs freshly isolated from cerebral small vessels (without culturing) of ˜3-month-old TgNotch3R90C mice was performed. It was discovered that the Tg-VSMCs show significantly different transcriptomic profiling as compared to WT-VSMCs. To identify the upstream regulators and regulatory networks for these affected genes in the Tg-VSMCs, Ingenuity Pathway Analysis (QIAGEN) was performed. Strikingly, the vegf gene was identified as one of the most important upstream regulators for the affected genes in Tg-VSMCs. Other important upstream regulators that show tight connections with vegf are p38 MAPK and NF-kB which are also affected in Tg-VSMCs as compared to WT-VSMCs (FIG. 9). More specifically, FIG. 9 depicts an ingenuity pathway analysis of RNAseq data. Upstream regulators and regulatory networks for the affected genes in freshly isolated brain VSMCs from about 3-month-old female tGNotch3R90C mice. VEGF expression and production have been shown to be regulated by ERK and NF-kB signaling. ERK is the best representative molecule of p38 MAPK. In addition to reduced VEGF-A, both the ERK and NF-kB signaling activations were also decreased in the Tg-VSMCs isolated from cerebral small vessels of ˜3-month-old TgNotch3R90C mice, and SCF+G-CSF treatment increased the ERK and NF-kB signaling activations in the Tg-VSMCs (FIGS. 10A-10C). These findings further confirm that VEGF-A activation is downregulated in cerebral VSMCs with Notch3R90C mutation 7 months prior to degeneration of VSMCs (occurring at the age of 10 months in TgNotch3R90C mice), and that SCF+G-CSF treatment could ameliorate the downregulated VEGF-A activation in the Tg-VSMCs. FIGS. 10A, 10B and 10C depict reduced ERK and NF-kB signaling is restored by SCF+G-CSF treatment in the Tg-VSMCs isolated from the brains of about 3-month-old female mice. *p<0.5, n=4.

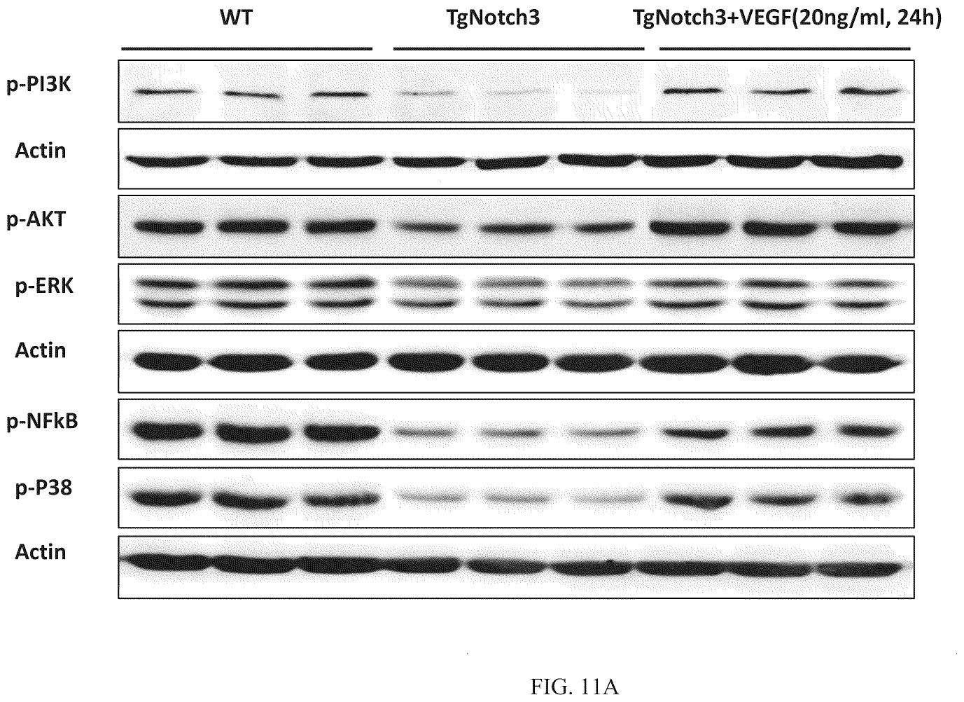

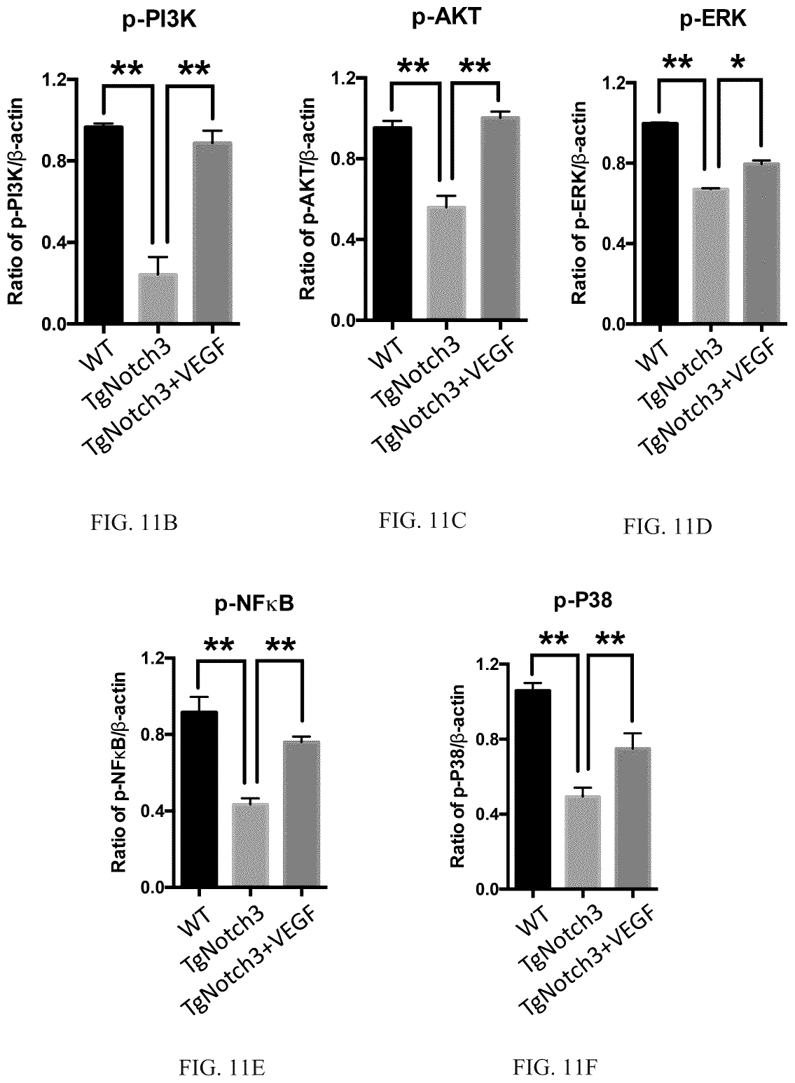

Restoration of affected cell signaling activation by VEGF treatment. In addition to ERK and NF-kB signaling, PI3K/AKT signaling is also involved in VEGF-associated cell signaling. P38 plays a key role in regulation of cell survival. After giving VEGF treatment (20 ng/ml) for 24 hours, decreased activations of PI3K/AKT, MEK/ERK, NF-kB and P38 in the Tg-VSMCs isolated from the brains of 3˜5-month-old TgNotch3R90C mice were significantly elevated (FIGS. 11A-11F). These findings, for the first time, have revealed the efficacy of VEGF treatment in ameliorating the impaired cell signaling activation that affects cell survival and metabolism in VSMCs with CADASIL-related mutations.

Results show that decreased VEGF production and decreased VEGF-related cell signaling activation are found in the mural cells of TgNotch3R90C mice at ˜3 months of age, which is ˜7 months earlier than VSMC degeneration and cerebrovascular dysfunction that happen at the age of 10 months in the TgNotch3R90C mice. The data above demonstrates, for the first time, that treatments targeting amelioration of the deficient VEGF have reparative effects in TgNotch3R90C mice.

Using brain sections from CADASIL patients, it was further confirmed the decreased blood vessel density in the brains of CADASIL patients. NOTCH3 mutation-caused blood vessel degeneration in the brain is the key pathological mechanism in CADASIL, and SCF+G-CSF-enhanced brain repair and cognitive recovery may be modulated through increasing blood vessel regeneration (i.e. angiogenesis). In the experiments above an angiogenic inhibitor was used to block the SCF+G-CSF-enhanced angiogenesis. This inhibitor is called bevacizumab or AVASTIN® brand Bevacizumab (Roche) which is an anti-VEGF monoclonal antibody to inhibit angiogenesis through neutralizing VEGF-A. VEGF-A is also known as VEGF, which has been proven to be the most potent proangiogenic factor for promoting angiogenesis

During carrying out the experiments, it was discovered that VEGF plays a key role in pathogenesis and development of CADASIL, and in mediating the SCF+G-CSF-enhanced cerebrovascular regeneration, brain repair and cognitive recovery in CADASIL mice (TgNotch3R90C mice). To block the SCF+G-CSF-enhanced angiogenesis, bevacizumab or AVASTIN® brand bevacizumab was injected before administering SCF+G-CSF in CADASIL mice (TgNotch3R90C mice). It was observed that pretreatment with bevacizumab (AVASTIN® brand bevacizumab), an angiogenic inhibitor which neutralizes VEGF-A, completely eliminated the SCF+G-CSF-enhanced cerebral blood vessel density, cognitive function recovery, vascular and neuronal structure regeneration, synaptogenesis and neurogenesis in TgNotch3R90C mice. It was also confirmed that SCF+G-CSF-enhanced endothelial cell (EC) proliferation and angiogenesis in TgNotch3R90C mouse brain-isolated ECs were also blocked by Avastin pretreatment. To further validate the effects of SCF+G-CSF in increasing VEGF production in the brains of TgNotch3R90C mice, brain samples were collected 24 hours after final injections of SCF+G-CSF treatment. Surprisingly, significant increases of VEGF protein levels were seen in the TgNotch3R90C mice treated with SCF+G-CSF. It was also discovered that VEGF protein levels in the brains of TgNotch3R90C mice were significantly decreased as compared to wild type control mice. These data suggest that SCF+G-CSF treatment repairs Notch3R90C mutation-damaged brain through the VEGF-A-mediated angiogenesis, which sheds new light on the mechanism underlying the SCF+G-CSF-enhanced brain repair in CADASIL. Importantly, this study provides novel insight into the involvement of VEGF in the pathogenesis of CADASIL and offers a novel molecular target to develop new treatments for CADASIL.

### Prophetic Example I

A human presents with CADASIL disease. Vascular endothelial growth factor (VEGF), functional isoforms thereof, or pharmaceutically acceptable salt forms thereof are administered intranasally to a subject in need thereof. VEGF is provided in a therapeutically effective amount to treat or ameliorate CADASIL disease. The patient's symptoms of CADASIL disease improve and the patients natural state of having CADASIL disease is altered or improved.

### Prophetic Example II

A human presents with CADASIL disease. SCF and G-CSF, functional isoforms thereof, or pharmaceutically acceptable salt forms thereof, are administered to a subject in need thereof. SCF and G-CSF are provided in a therapeutically effective amount to treat or ameliorate CADASIL disease. The total amount of VEGF in the patient increases. The patient's symptoms of CADASIL disease improve and the patients natural state of having CADASIL disease is altered or improved.

The entire disclosure of all applications, patents, and publications cited herein are herein incorporated by reference in their entirety. While the foregoing is directed to embodiments of the present disclosure, other and further embodiments of the disclosure may be devised without departing from the basic scope thereof.

1. A method for preventing, treating or ameliorating cerebral autosomal dominant arteriopathy with subcortical infarcts and leukoencephalopathy (CADASIL), comprising:
administering in advance of vascular smooth muscle cell (VSMC) degeneration and cerebrovascular dysfunction, a vector containing a nucleotide sequence and/or an amino acid sequence having at least 90% sequence identity to at least one of SEQ ID NO: 1, SEQ ID NO: 2, SEQ ID NO:3, SEQ ID NO:4; SEQ ID NO:5, or SEQ ID NO:6 into the vascular smooth muscle cells (VSMCs) of small arteries and pericytes of capillaries of a brain of a subject in need thereof, to provide a vascular endothelial growth factor (VEGF), or functional isoforms thereof.

2. The method of claim 1, wherein VEGF, or functional isoforms thereof, comprise one or more of VEGFa, VEGFb, VEDFc, or combinations thereof.

3. (canceled)

4. The method of claim 1, wherein administering VEGF, or functional isoforms thereof, comprises administering a granulocyte colony-stimulating factor (G-CSF) polypeptide in combination with a stem cell factor (SCF) polypeptide, in an amount sufficient to increase VEGF in a subject in need thereof.

5. The method of claim 1, wherein administering VEGF, or functional isoforms thereof, comprises administering: 1) a cDNA that encodes one or more proteins comprising VEGFa, VEGFb, VEGFc, a VEGF isoform, or combinations thereof; or 2) one or more nucleic acid sequences that encode one or more proteins comprising a VEGFa, VEGFb, VEGFc, a VEGF isoform, or combinations thereof.

6. A method of improving neurological function and enhancing cerebrovascular maintenance and regeneration in a mammal suffering from cerebral autosomal dominant arteriopathy with subcortical infarcts and leukoencephalopathy (CADASIL), comprising: administering a vector containing a nucleotide sequence and/or an amino acid sequence having at least 90% sequence identity to at least one of SEQ ID NO: 1, SEQ ID NO: 2, SEQ ID NO:3, SEQ ID NO: 4; SEQ ID NO:5, or SEQ ID NO:6 into the vascular smooth muscle cells (VSMCs) of small arteries and pericytes of capillaries of said mammal, to provide vascular endothelial growth factor (VEGF), or a functional isoform thereof, to a mammal subject in need thereof.

7. The method of claim 6, wherein a pharmaceutically acceptable composition comprising a granulocyte colony-stimulating factor (G-CSF) polypeptide is administered in combination with a stem cell factor (SCF) polypeptide, in an amount effective in increasing VEGF in a subject in need thereof.

8. The method of claim 6, wherein an improvement in neurological function is characterized by improved cognitive function.

9. The method of claim 6, wherein an improvement in neurological function and an enhancement of cerebrovascular maintenance and regeneration is characterized by slowing a progress of CADASIL, increased blood vessel density, increased angiogenesis, increased vascular cell proliferation, restored cerebral blood vessels, restored neurogenesis, increased density of axons, increased density of dendrites, increased density of synapses, improved spatial learning, improved memory, enhanced neurostructural regeneration, enhanced synaptogenesis and enhanced neurogenesis.

10. The method of claim 6, wherein the subject is a human or a mouse.

11. The method of claim 6, wherein administering further comprises targeting VEGF with a composition in an amount sufficient to increase VEGF and/or VEGF-regulated angiogenesis and cell survival signaling.

12. A method of improving neurological function and cerebral blood flow and enhancing cerebrovascular maintenance and regeneration in a mammal suffering from cerebral autosomal dominant arteriopathy with subcortical infarcts and leukoencephalopathy (CADASIL), comprising administering an effective amount of a composition comprising a stem cell factor (SCF) polypeptide, alone or in combination with a granulocyte colony stimulating factor (G-CSF) polypeptide.

13. The method of claim 12, wherein the composition comprises a SCF polypeptide alone.

14. The method of claim 12, wherein an improvement in neurological function is characterized by improved sensorimotor skills and coordination and ameliorated depression and anxiety.

15. The method of claim 12, wherein an effective amount is an amount sufficient to increase a VEGF and/or a VEGF-regulated angiogenesis and cell survival signaling.

16. The method of claim 12, wherein restored neurogenesis and a density of axons, dendrites, and synapses.


SEQUENCE LISTING (6 sequences)

SEQ ID NO: 1 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 2 - protein, 215 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 3 - protein, 209 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRPCGPCSERRKHLFVQDPQTCKCSCKNTDSRCKARQLELNERTCRCDKPRR

SEQ ID NO: 4 - protein, 214 aa, synthetic construct
  REGION 1..214: note = Synthetic sequence
MNFLLSWVHWTLALLLYLHHAKWSQAAPTTEGEQKSHEVIKFMDVYQRSYCRPIETLVDIFQEYPDEIEYIFKPSCVPLMRCAGCCNDEALECVPTSESNITMQIMRIKPHQSQHIGEMSFLQHSRCECRPKKDRTKPEKKSVRGKGKGQKRKRKKSRFKSWSVHCEPCSERRKHLFVQDPQTCKCSCKNTDSRCKARQLELNERTCRCDKPRR

SEQ ID NO: 5 - protein, 207 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 6 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS